(19) United States
(12) Patent Application Publication
(10) Pub. No.: US 2018/0206711 A1
(43) Pub. Date: Jul. 26, 2018

(54) SYSTEM FOR A MINIMALLY-INVASIVE, OPERATIVE TREATMENT

(71) Applicant: Boston Scientific Scimed, Inc., Maple Grove, MN (US)

(72) Inventor: Gregory Piskun, Morganville, NJ (US)

(21) Appl. No.: 15/875,295

(22) Filed: Jan. 19, 2018

Related U.S. Application Data
(60) Provisional application No. 62/448,859, filed on Jan. 20, 2017.

(51) Int. Cl. A61B 1/01; A61B 1/31; A61B 1/015; A61B 17/32; A61B 17/29; A61B 1/00; A61B 1/018; A61B 1/005

(52) U.S. Cl. CPC A61B 1/01; A61B 1/31; A61B 1/015; A61B 1/0058; A61B 17/29; A61B 1/00135; A61B 1/018; A61B 17/320016

(57) ABSTRACT

A system for performing minimally invasive procedures in a body lumen of a patient including a flexible catheter having a first lumen and a second lumen, the first and second lumens terminating in a distal opening at a distalmost end of the catheter. First and second flexible guides are separate components from the catheter and removably insertable into the flexible catheter and movable axially through the lumen of the flexible catheter. The guides have a channel extending therethrough configured and dimensioned to receive an endoscopic tool for axial movement therein. The flexible guides have a longitudinal axis and a tube distal portion movable to a pre-shaped curved position with respect to the longitudinal axis when exposed from the flexible catheter.

### CROSS REFERENCE TO RELATED APPLICATIONS

The present application claims the benefit of priority under 35 U.S.C § 119 to U.S. Provisional Patent Application Ser. No. 62/448,859, filed on Jan. 20, 2017, which is incorporated by reference in its entirety for all purposes.

### BACKGROUND

### Field of the Invention

This application relates to minimally invasive apparatus and methods for performing surgical procedures within body lumens of a patient such as the gastrointestinal system.

### Description of the Related Art

Endoscopic procedures involving the gastrointestinal system offer advantages over conventional surgery in that they are less invasive, may provide visualization and reduce the length and expense of a hospital stay.

It is advantageous in minimally invasive procedures to achieve triangulation of instrumentation within the body lumen to more effectively simulate open procedures. Such procedures need to accommodate a wide variety of minimally invasive surgical instruments for independent movement while avoiding interfering with visualization of the body space and target tissue. Additionally, it is also advantageous to provide endoscopic technology for organizing the endoscope and instruments in a manner that can maximize the working space for treatment. This improves the ability to manipulate the instruments and endoscope in a minimally-invasive manner from outside the body. It is recognized to be advantageous to have a working space that has tips of the instruments as far as practical from the target tissue to improve the maneuverability of the instruments and provide additional flexibility in approaching and visualizing the target tissue, thereby providing more operating room for selecting a trajectory of the instruments toward the target tissue that is, for example, at least substantially perpendicular to the plane of dissection of the target tissue.

In view of the above, one of skill in the art of endoscopic surgical treatments would appreciate the technology taught herein which provides an organization of the endoscope and instruments to maximize the working space and maneuverability, allowing for a maximum flexibility in approaching and visualizing the target tissue. It should be appreciated that having such improvements would reduce the technical complexity, doing so at a low cost, while using a system introduced in a manner that does not substantially disrupt conventional endoscopy.

### SUMMARY

The teachings provided herein are generally directed to improved methods and devices for operatively treating disorders endoscopically in a stable, yet dynamic operative environment, and in a minimally-invasive manner. Embodiments taught herein provide, among other aspects, an increase in distance between tool ports and the target tissue to improve maneuverability and triangulation of the tools with respect to the target tissue, as well as a larger field of view.

In accordance with one aspect, a system for performing minimally invasive procedures in a body lumen of a patient is provided comprising a flexible catheter having a proximal end, a distal end, a first lumen and a second lumen, the first lumen terminating in a first distal opening at a distalmost end of the catheter and the second lumen terminating in a second distal opening at a distalmost end of the catheter. The first and second distal openings are at the terminal end of the flexible catheter. A first flexible guide is a separate component from the catheter and is removably insertable into the flexible catheter and movable axially through the first lumen of the flexible catheter. The first flexible guide has a first channel extending therethrough configured and dimensioned to receive a first endoscopic tool for axial movement therein, and terminating in a first opening. The first flexible guide has a first longitudinal axis and a tube distal portion movable to a pre-shaped curved position with respect to the first longitudinal axis when exposed from the flexible catheter. A second flexible guide is a separate component from the catheter and is removably insertable into the flexible catheter and movable axially through the second lumen of the flexible catheter. The second flexible guide has a second channel extending therethrough configured and dimensioned to receive a second endoscopic tool for axial movement therein, and terminating in a second opening. The second flexible guide has a second longitudinal axis and a tube distal portion movable to a pre-shaped curved position with respect to the second longitudinal axis when exposed from the flexible catheter.

In some embodiments, the first lumen is in a first flexible tube and the second lumen is a second flexible tube. In some embodiments, the first and second flexible tubes are fixed at a proximal portion to the flexible catheter and configured to float within the flexible catheter such that at least an intermediate portion of the first flexible tube moves radially within the first lumen. In some embodiments, the first flexible tube and second flexible tube are unattached to the flexible catheter at a distal end.

In some embodiments, the first and second flexible guides have different indicators to differentiate the guides.

In some embodiments, the first and second flexible guides are removably insertable through respective first and second proximal ports of the flexible catheter and are independently rotatable and axially movable within the catheter, the first proximal port communicating with the first lumen and the second proximal port communicating with the second lumen. In some embodiments, one or both of the first flexible guide and second flexible guide includes a valve at a proximal portion to accommodate the respective endoscopic tool without losing insufflation. The first and second flexible guides are preferably unattached to the catheter during manipulation by a user from the proximal region, the proximal region protruding proximally from the proximal end of the catheter. In some embodiments, the distal portions of the first and second flexible guides are in a straighter condition positioned within the confines of the catheter and automatically return to the preset curved position when exposed from the catheter. The first and second flexible guides can in some embodiments be retractable proximally into the catheter.

In some embodiments, the first and second flexible guides are composed of shape memory material.

In some embodiments, the system further comprises an endoscope positionable in the flexible catheter and movable independently of the first and second flexible guides. In some embodiments, the catheter has a third lumen to receive the endoscope, wherein the first and second lumens lie below a longitudinal centerline of the catheter and the third lumen extends above the centerline. In some embodiments, the endoscope floats within the catheter to increase the flexibility of the catheter.

In some embodiments, a diameter of a proximal region of the first flexible guide is greater than a diameter of a distal region to provide a stop for distal insertion of the first flexible guide within the first lumen.

In some embodiments, a first seal is provided within the first lumen and a second seal is provided within the second lumen wherein the first and second seals limit gas leakage in a space between the flexible guides and the respective lumen.

A third lumen to receive the endoscope, if provided, can have a third seal to limit gas leakage in a space between the endoscope and the third lumen.

In some embodiments, a blocking member is provided external of the catheter to limit gas leakage.

In some embodiments, a first tubular support and a second tubular support are positioned within the flexible catheter, wherein the first flexible tube is unattached to the flexible catheter at a distal end so the first flexible tube telescopes within the first tubular support as the flexible catheter is bent a sufficient amount and wherein the second flexible tube is unattached to the flexible catheter at a distal end so the second flexible tube telescopes with respect to the second tubular support as the catheter is bent a sufficient amount.

In accordance with another aspect, a kit for performing minimally invasive procedures in a body lumen of a patient is provided comprising: a) first flexible guide removably insertable into a flexible catheter and movable axially through the flexible catheter, the first flexible guide having a first channel extending therethrough configured and dimensioned to receive a first endoscopic tool for axial movement therein, the first channel terminating in a first opening, the first flexible guide having a first longitudinal axis and a tube distal portion movable to a pre-shaped curved position with respect to the first longitudinal axis when exposed from the flexible catheter; and b) a second flexible guide removably insertable into the flexible catheter and movable axially through the flexible catheter, the second flexible guide having a second channel extending therethrough configured and dimensioned to receive a second endoscopic tool for axial movement therein, the second channel terminating in a second opening, the second flexible guide having a second longitudinal axis and a tube distal portion movable to a pre-shaped curved position with respect to the second longitudinal axis when exposed from the flexible catheter, the second flexible guide being different than the first flexible guide.

In some embodiments, the first and second flexible guides are different in that the pre-shaped curve of the first flexible guide is a different configuration than the pre-shaped curve of the second flexible guide. In some embodiments, the pre-shaped curve of the first flexible guide has a greater radius of curvature than the pre-shaped curve of the second flexible guide.

In accordance with another aspect, a system for performing minimally invasive procedures in a body lumen of a patient is provided, the system comprising an endoscope having a working channel, an overtube having an opening dimensioned to receive the endoscope and an elongated channel extending from the overtube and extending external of an outer wall of the endoscope, the elongated channel dimensioned to receive an instrument therethrough.

In some embodiments, the channel and overtube are monolithic; in other embodiments they are separate components attached together.

### BRIEF DESCRIPTION OF THE FIGURES

FIG. 1 is perspective view of one embodiment of a system for operatively treating disorders endoscopically, showing the catheter and two flexible guides (tool channels).

FIG. 2A illustrates insertion of an endoscope through the colon in accordance with one method of the present disclosure.

FIGS. 2B and 2C illustrate the catheter of FIG. 1 being inserted into the colon.

FIG. 3 illustrates the catheter of FIG. 1 positioned in the colon and the endoscope retracted to a substantially flush position with the catheter.

FIG. 4A is a perspective view of the system of FIG. 1 shown prior to insertion of the flexible guides through the ports of the catheter, and further showing in phantom the straightened position of the flexible guides when positioned within the catheter.

FIG. 4B is a perspective view similar to FIG. 4A showing an alternate embodiment of the flexible guides.

FIG. 4C is a perspective view similar to FIG. 4A showing another alternate embodiment of the flexible guides.

FIG. 4D is a perspective view similar to FIG. 4A showing another alternate embodiment of the flexible guides.

FIG. 4E is a perspective view of one example of a kit containing two differently configured flexible guides.

FIG. 5 is a perspective view illustrating the flexible guides inserted into the catheter of FIG. 1.

FIG. 6 is a perspective view illustrating an alternate embodiment of the flexible guides inserted into the catheter of FIG. 1.

FIG. 7 is a perspective view showing the tool channels of FIG. 4A advanced from the catheter to assume their pre-curved position.

FIG. 8 is a perspective view showing the tool channels of FIG. 4C advanced from the catheter to assume their pre-curved position.

FIG. 9A is a perspective view similar to FIG. 7 showing an endoscopic instrument advanced from each of the flexible guides.

FIG. 9B is a perspective view similar to FIG. 9A showing the endoscopic instruments further advanced from each of the flexible guides.

FIGS. 10A and 10B are side and front views of the flower retractor configured for insertion through a flexible guide;

FIG. 11A is a front view of the system of FIG. 1 showing two flexible guides extending from the catheter.

FIG. 11B is a front view of the system similar to FIG. 11A showing two instruments extending from the two flexible guides to achieve instrument triangulation.

FIG. 12 is a perspective view of the distal end of the outer tube (catheter) of an alternate embodiment of the system showing a floating channel therein.

FIGS. 13A-13C are perspective views showing movement of the floating channel within the outer tube (catheter) in accordance with another embodiment of the floating system.

FIGS. 14A and 14B are transverse cross-sectional views through the outer tube (catheter) showing radial movement of an intermediate portion of the floating channels within the lumen of the outer tube.

FIG. 15A is a cross-sectional view illustrating bending of the outer tube (catheter) and movement of the floating channels of FIGS. 13A-13C.

FIG. 15B is a perspective view of the proximal end of the flexible channels showing the proximal tubes.

FIG. 16 is a side perspective view of the distal portion of the system of FIG. 15A showing the effect of bending of the outer tube and movement of the floating channels.

FIG. 17 is a perspective view of alternate embodiment of the catheter having a sealing cuff.

FIG. 18 is a perspective view of another alternate embodiment of the catheter having internal seals.

FIG. 19A is a perspective view of alternate embodiment of the system having an endoscope, overtube and an external channel.

FIG. 19B is an exploded view of the system of FIG. 19A.

### DETAILED DESCRIPTION

The systems and methods disclosed herein are generally directed to improved methods and devices for operatively treating endoscopically various body regions, such as the gastrointestinal tract, to maximize space for a tool (instrument) and an endoscope to each be maneuvered independently to visualize a target tissue and treat the target tissue from outside the patient in a minimally invasive manner. Embodiments taught herein can provide, among other aspects, an increase in distance between tool ports and the target tissue to enhance the independent maneuverability and triangulation of each of the tools with respect to the target tissue. This increase in distance can also provide a way of obtaining a larger field of view. The systems taught herein, for example, can (i) provide a flexible passageway for multiple surgical tools and instruments, such as an endoscope and graspers to be passed from outside the body towards the target tissue; (ii) organize and/or constrain tools in the working space within the body lumen; and (iii) enable control over the position and orientation of the instruments in the working space from outside the body.

In some embodiments disclosed herein, an articulating endoscope is inserted through a channel of the catheter; in other embodiments the system is backloaded over a flexible endoscope, such as a conventional colonoscope. In some embodiments, the flexible endoscope is inserted to a position adjacent the target tissue and then the catheter is advanced over the endoscope so the openings in the catheter enable the tool channels and tools to be advanced to the target tissue.

Although the system is described herein for use in the GI tract, this is described by way of example as the system can also be used in other body regions such as in the biliary tree, bronchi, trachea, uterus, ovarian tubes, vessels, etc.

In the systems disclosed herein, a flexible tube (also referred to herein as a flexible guide or tool channel) is selectively inserted through the lumen (or channel) of the catheter and acts as a guide for endoscopic working instruments which are selectively inserted therethrough. That is, the flexible tube is first inserted into the lumen or channel of the catheter and then the endoscopic instrument is inserted through a lumen in the respective flexible tube, exiting a distal opening in the flexible tube. The flexible tube has a preformed curved at a distal end which automatically assumes the curved position when exposed from the catheter so it can curve toward the target tissue. The curving and maneuverability of the flexible tubes control the positioning and orientation of the endoscopic instruments, and therefore the endoscopic instruments need not be provided with a pre-curved tip or articulating mechanisms. The flexible tubes can have various degrees of curvature, various lengths of curved tips, and various curved configurations, e.g., C-shaped, S-shaped, etc. This enables the user to select prior to and during the surgical procedure the desired curved tool channel. This is described in more detail below. The tool channels can in some embodiments be provided in a variety of kits, also described below.

In preferred embodiments, the systems disclosed herein are used in conjunction with insufflation of the body lumen, such insufflation expanding the body lumen space to create more working space around the target tissue to create a larger area for tissue access and treatment. This larger space increases the maneuverability of the instruments as the distances between the instruments and target tissue within the body lumen are increased.

The methods, devices, and systems taught herein are used for minimally-invasive procedures which reduces trauma to the patient, speeds the healing process, minimizes tissue damage, and reduces the length and expense of a hospital stay. Tissue damage, or the risk thereof, can be minimized or avoided, for example, where a procedure is designed to minimize or avoid unnecessary tissue contact that may otherwise be associated with a procedure. The gentle procedures taught herein, for example, are directed to preserving tissue during a gastrointestinal or other surgery.

The systems disclosed herein also enable triangulation to be achieved in a minimally invasive procedure. Tissue triangulation, wherein the tissue is triangulated between two endoscopic instruments, enhances access and maneuverability.

The outer tube (catheter) of the system can be a multi-luminal tube, so a separate lumen, or a separate tube forming a lumen, accommodates the endoscope and separate lumens, or separate tubes forming lumens, accommodates the individual tool channels, and during the use of the system, the tool channel serves as a guide through which a tool (endoscopic instrument) can be inserted and manipulated in a treatment of a target tissue, e.g., in the gastrointestinal tract of the patient. The length of the channel is sufficient so it can extend out the proximal end of the outer tube for manipulation by the user. The tool channels, due to their pre-curved distal end, bend at a distal end when exposed from the outer tube so they angle away from the longitudinal axis and can be directed toward the target tissue.

A variety of tools (endoscopic instruments) can be inserted through the tool channels including for example a grasper, a forceps, a snare, a scissor, a knife, a dissector, a clamp, an endoscopic stapler, a tissue loop, a clip applier, a suture-delivering instrument, an energy-based tissue coagulator or cutter, etc. It should be appreciated that the terms “tool” and “instrument” can be used interchangeably. As can be appreciated, when the tool channel bends in a manner described herein due to its pre-bend, it bends the tool positioned therein.

Although in the Figures two tool channels are illustrated with each catheter, it should be appreciated that a system with more than two tool channels or with only one tool channel can also be utilized. Additionally, the endoscope can have a working channel extending therein for insertion of one or more working instruments such as a grasper or dissector.

The outer tube can comprise a polymer, which in some embodiments can have an embedded wire reinforcement such as a mesh, a braid, a helical coil or any combination thereof composed of, for example, stainless steel. The outer tube is flexible, elastically bendable, but sufficiently stiff torsionally to transmit torque from the handle or proximal end of the system to the distal end of the system.

The outer tube can be connected at a distal end to a ring or distal coupler (cap) which can form the terminal end of the outer tube. The coupler can have portals formed therein for a desired orientation and positioning of the endoscope and tool channels for the tools, such that the endoscope and tool channels (and tools) are organized relative to each other in a predetermined manner to achieve a particular function, such as an increase in working space and a better view of a plane of dissection.

In some embodiments, the catheter is slidably positioned over an endoscope, such as a colonoscope, during use. In these embodiments, the endoscope is first inserted to a position adjacent the target tissue and then the multi-lumen tube or catheter is advanced over the endoscope, with the endoscope received within the endoscope receiving lumen (channel) of the outer tube or catheter. The method can include inserting the multi-luminal tube into an overtube, cover, or sheath.

In some embodiments, the systems can include a multi-lumen catheter having at least two tool channels for manipulating tools and an endoscope, each of the two working channels having six degrees of freedom that are independent from each other and the endoscope. The ability to independently manipulate the endoscope and tool channels allows, for example, one instrument to retract the tissue or lesion away or substantially perpendicular to another instrument, for example, the dissecting instrument, while independently optimizing the endoscope's position and, hence, the view of the treatment area. This would facilitate the removal of tissue with clear margins. The tool channels can manipulate the tools with several degrees of freedom, six degrees of freedom in some embodiments, providing a greatly enhanced maneuverability in the working area when compared to current state-of-the-art systems. The tool channels and tools can be maneuvered within the working space to enable tissue triangulation.

The systems taught herein can provide for organizing the orientation of the tool channels in order to further facilitate improving the flexibility of the system. In some embodiments, for example, the coupler can be used to organize the tools and endoscope in a particular arrangement to facilitate a particular positioning of the tools as they emerge from the outer tube into the working space. The proximal end of the outer tube can also have respective openings for each of the tool channels and for the endoscope, and these openings can be, for example, a part of a handle coupler, or the handle itself, for insertion of the tool channels. The endoscope and tool channels (and tools inserted through the tool channels) can be manipulated from outside the patient.

In some embodiments, floating systems can be provided which have a floating channel, a floating endoscope, multiple floating channels, or a combination thereof, as described below. The floating enables the channel substantial freedom to move within the outer tube during operation.

In some embodiments, the port can be substantially larger than the scope, such that the scope can slide axially, as well as move side-to-side, align its central axis parallel to the central axis of the outer tube, or perhaps, misalign its central axis to not be parallel to the central axis of the outer tube.

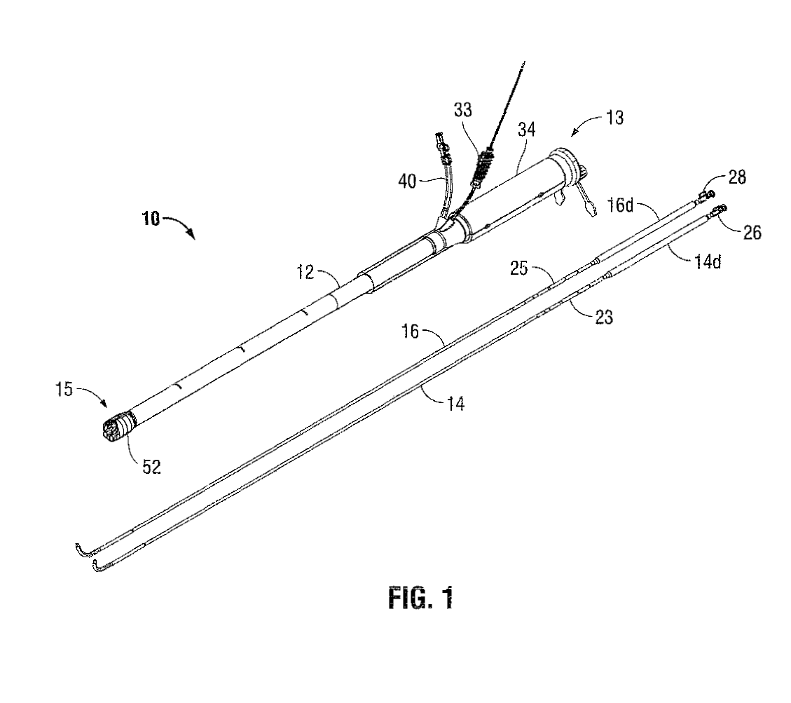

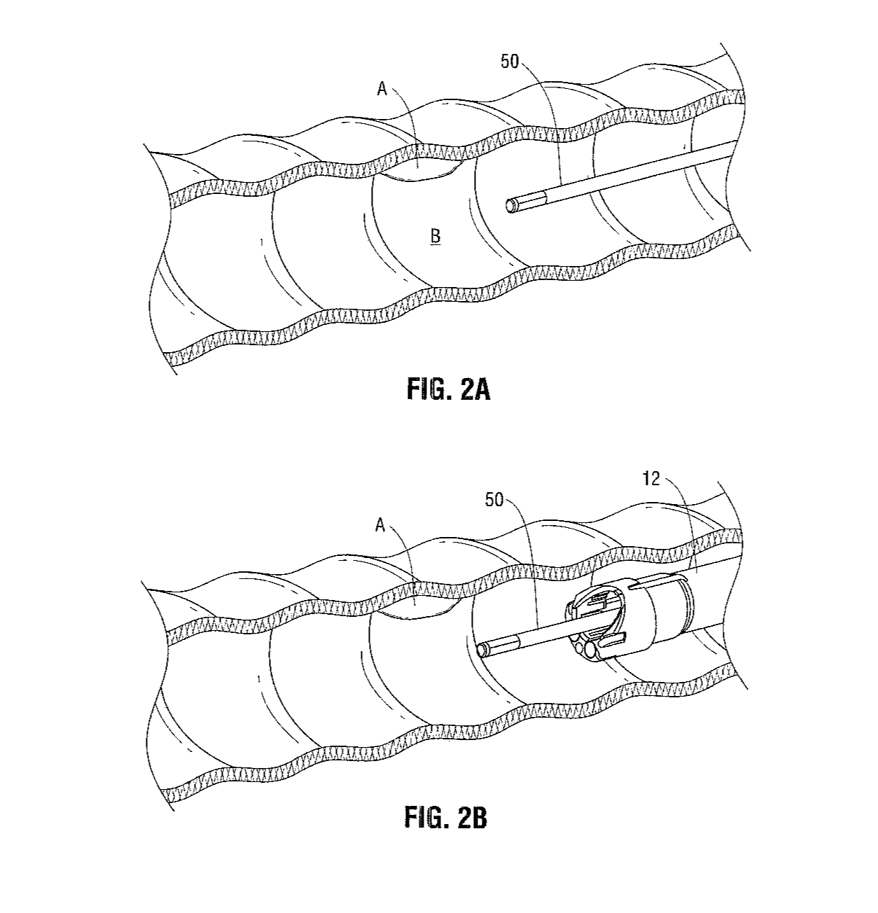

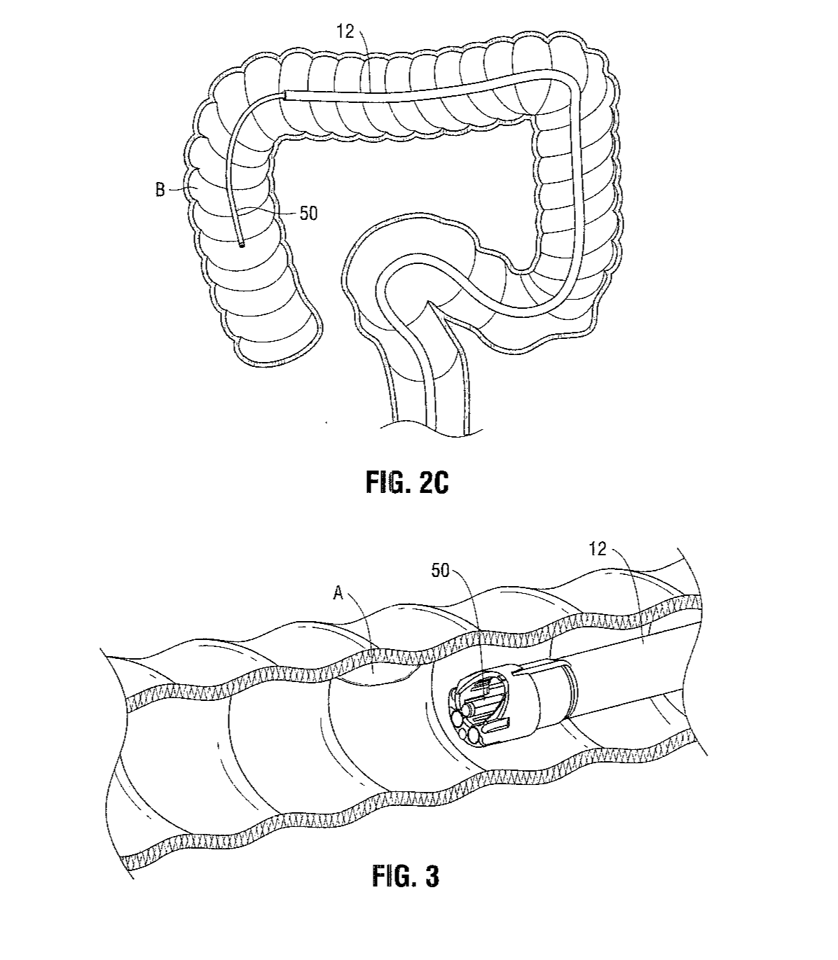

FIGS. 2A-2C illustrate use of a system disclosed herein for treating a lesion in the ascending colon by way of example. FIG. 2A illustrates insertion of an endoscope 50 to visually locate the target lesion, e.g., lesion A, in a portion of the ascending colon B. FIG. 2B illustrates an embodiment of the multi-lumen-catheter system, e.g. catheter 12 of FIG. 1 described below, guided to the target tissue A using the endoscope 50 as a guide for the positioning of the system in the treatment of the target tissue A. As can be appreciated, the multi-lumen catheter 12 is advanced over the endoscope 50 as shown in FIG. 2B. Alternatively, the catheter 12 can be first backloaded over the endoscope 50, and the catheter 12 and endo scope 50 inserted together, with the endoscope protruding from the catheter during insertion. In either insertion method, the catheter and endoscope are advanced through the colon B (FIG. 2C) to the target site and the catheter and endoscope are preferably moved relative to each other, i.e., the endoscope is retracted and/or the catheter is advanced, so the distal ends of the endoscope and catheter are preferably substantially flush as shown in FIG. 3. By the endoscope tip terminating adjacent the distal end of the catheter 12, maneuverability of the tool channels is enhanced. However, note that in some embodiments, the endoscope 50 can be advanced beyond the catheter distal end and articulated in the working space.

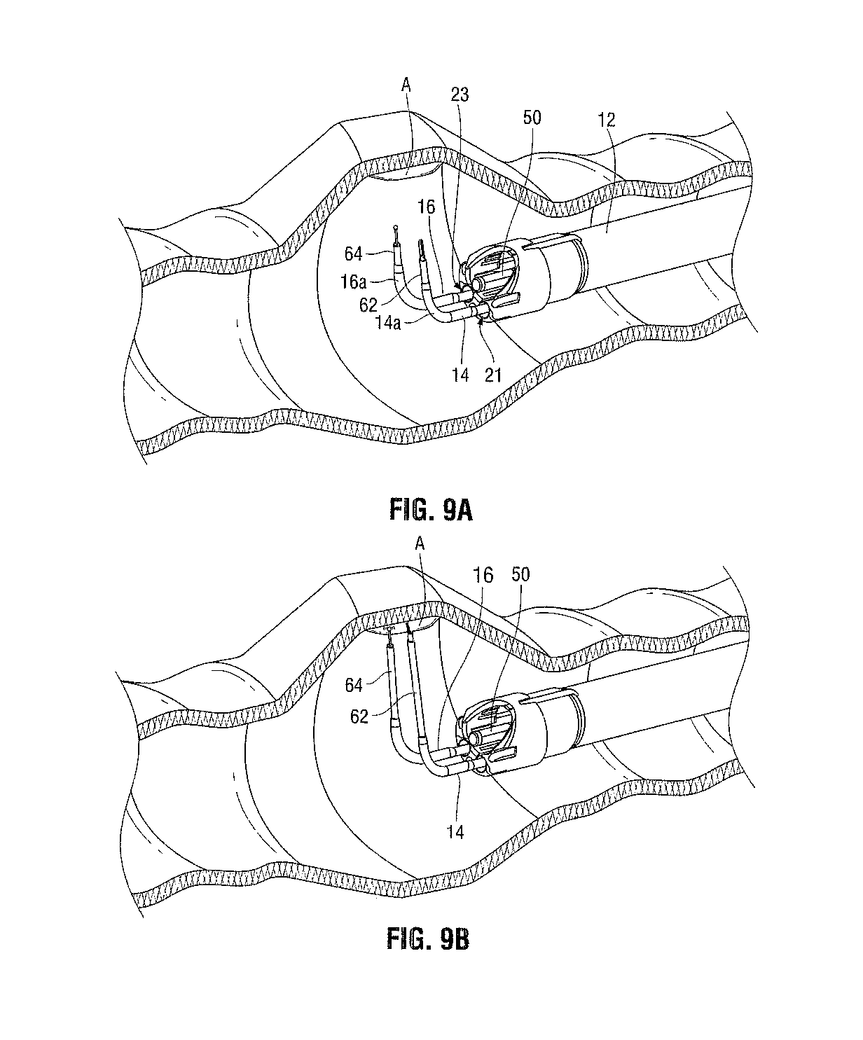

FIGS. 9A and 9B illustrate the versatility of the system, showing examples of the tools utilized to remove the lesion A from the colon-tool 64 to excise the lesion A from an independently chosen first angle, while tool 62 can be used to grasp the lesion A from an independently chosen second angle and endoscope 50 can be used to view the lesion A. As described below, the different angling of the tools 62, 64 advantageously achieves tissue triangulation to facilitate access, maneuverability and removal of the lesion. After the excision of the lesion A from the colon wall by the dissection tool 64, a tissue defect remains which can be closed for example by removal of the tool 64 and insertion of a tool for closure of the lesion. The lesion can be closed by various methods such as mechanical (e.g., clips, staples or structures), glue, electrosurgical energy, etc.

The endoscope and tools can be maneuvered independently, for example, to access the lesion at a greater range of angles. This increased maneuverability can improve the view of the lesion and ability to manipulate and dissect the lesion. For example, a grasper can be advanced out of the tool channel into the working space towards the polyp, grasp the polyp and retract the tissue to expose the base of the polyp for dissection by a dissection tool inserted through another tool channel of the multi-channel systems taught herein. In such embodiments, a dissection tool can be advanced through a channel at the base of the polyp and dissect the polyp's base where it attaches to the lumen wall, while the position of the endoscope provides a close view of the base of the polyp to help identify the desired margin for dissection.

The lesion can include, for example, a perforation, a tissue pathology a polyp, a tumor, a cancerous tissue, a bleed, a diverticuli, an ulcer, an abnormal vessel, or an appendix.

As described herein, the tool channels are configured to control the trajectory and position of instruments such as forceps in the working space. In some embodiments, a tool channel can be removed from, or inserted through, the outer tube of the system, alone or inside an additional channel that may be used as a guide.

In some embodiments, two inner tubes can be positioned adjacent to the inner surface of the outer tube to provide, effectively, three separate channels. The two inner tubes can function as two independent lumens to receive tool channels while the space between these first two inner tubes and the outer tube functions as a third channel. The third channel can be substantially larger than the other two channels. Each of the first two tool channels can have, for example, an inner diameter ranging from about 2 mm to about 6 mm, or about 3 mm to about 5 mm, or any range therein. In some embodiments, the diameter of the first two tool channels can be about 4 mm. Each of the inner tubes can be designed to accommodate a tool channel for passage of an endoscopic tool that includes, for example, forceps, graspers, clip applier, dissectors, snares, electrical surgical probes, or loops. In some embodiments, the largest diameter channel can be the lumen for the endoscope. In other embodiments, the endoscope is inserted within the space between the two channels, defined above as the third channel.

The channel for accommodating the endoscope can be designed to have an inner diameter, for example, ranging from about 5 mm to about 15 mm, from about 6 mm to about 12 mm, from about 11 mm to about 14 mm, from about 5 mm to about 10 mm, from about 8 mm to about 13 mm, or any range therein in 1 mm increments. The inner tubes can comprise any suitable material known to one of skill to be useful for the purposes set-forth herein, as well as composites thereof. For example, the inner tubes can comprise a fluoropolymer such as TEFLON for lubricity to ease tool or endoscope passage and movements. Other materials that may be used include, for example, polyethylene, polypropylene, PEBAX, nylon, polyurethane, silicone, and composites thereof, each of which may also be used with a lubricant coating. The tubes may also comprise a metallic wire reinforcement such as a braid, mesh or helical coil, each of which may be embedded in the tube.

Turning now to the drawings, wherein like reference numerals identify similar or like components throughout the several views, FIG. 1 illustrates one embodiment of the minimally invasive system of the present disclosure, designated generally by reference numeral 10. System 10 includes a multi-lumen catheter or tubular member 12 configured to receive one or more tool channels (also referred to herein interchangeably as flexible tubes, flexible instrument guides or flexible guides). FIG. 1 shows two tool channels 14 and 16, it being understood that in some embodiments, only one tool channel can be utilized and in other embodiments more than two tool channels can be utilized, with the catheter provided with a sufficient number of lumens to accommodate the tool channels. The tool channels 14, 16 can be packaged as a kit with the catheter 12 as shown in FIG. 1. Alternatively, the tool channels 14, 16 can be packaged separately. In other embodiments, the tool channels are packaged already inside the lumens of the catheter 12. Each tool channel 14, 16 has a lumen (channel) to slidably and preferably removably receive an endoscopic instrument (tool) therethrough.

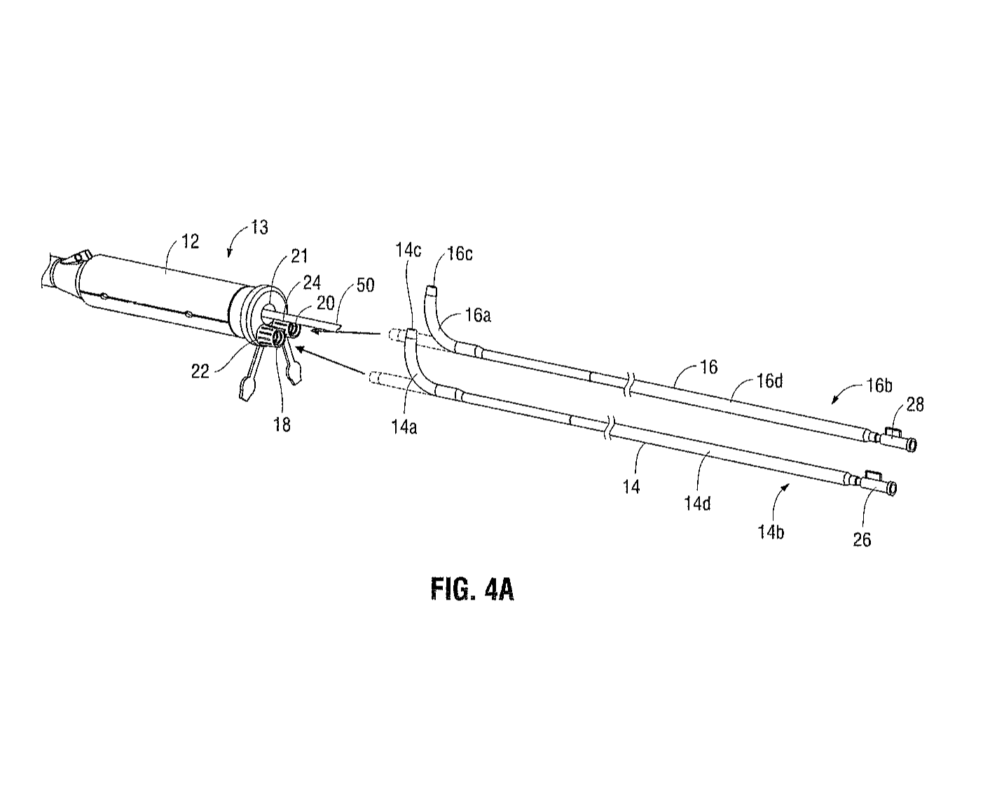

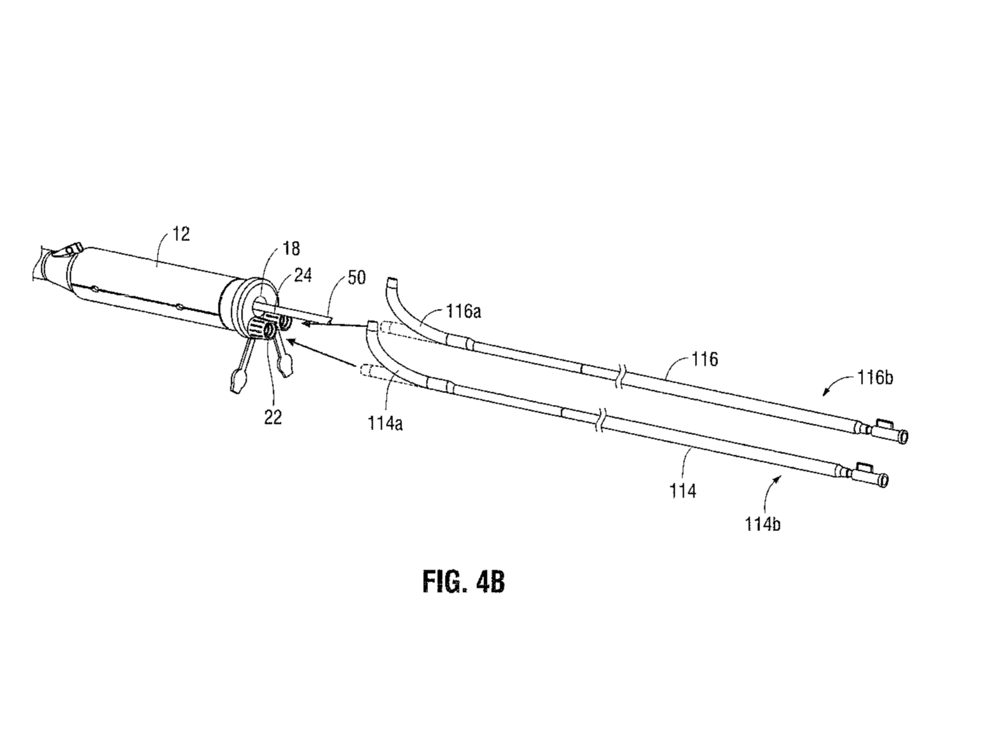

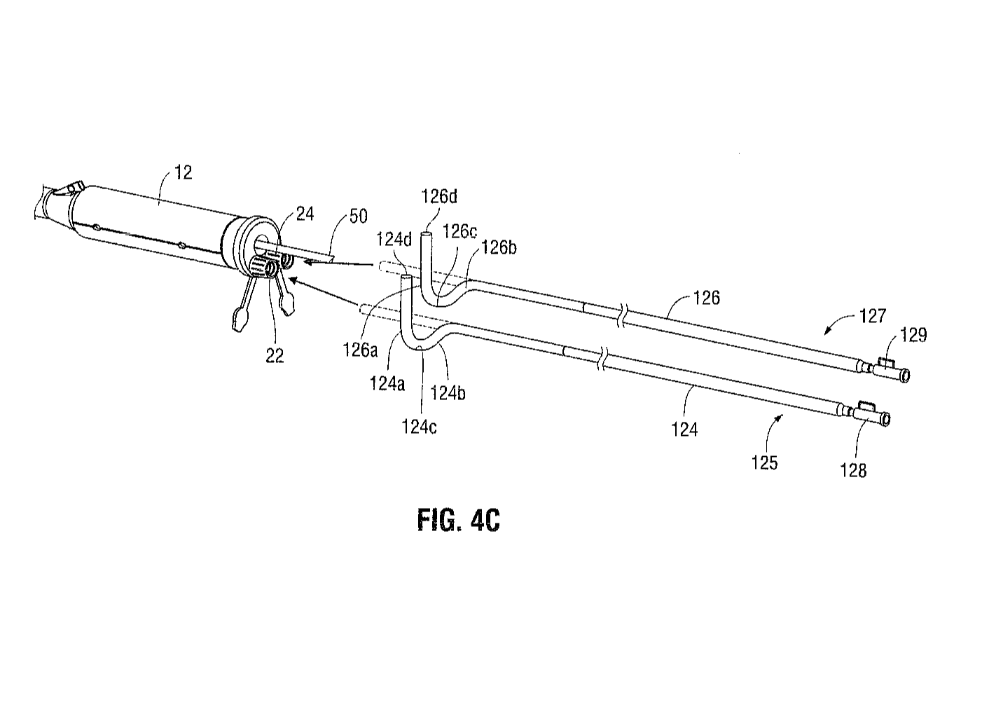

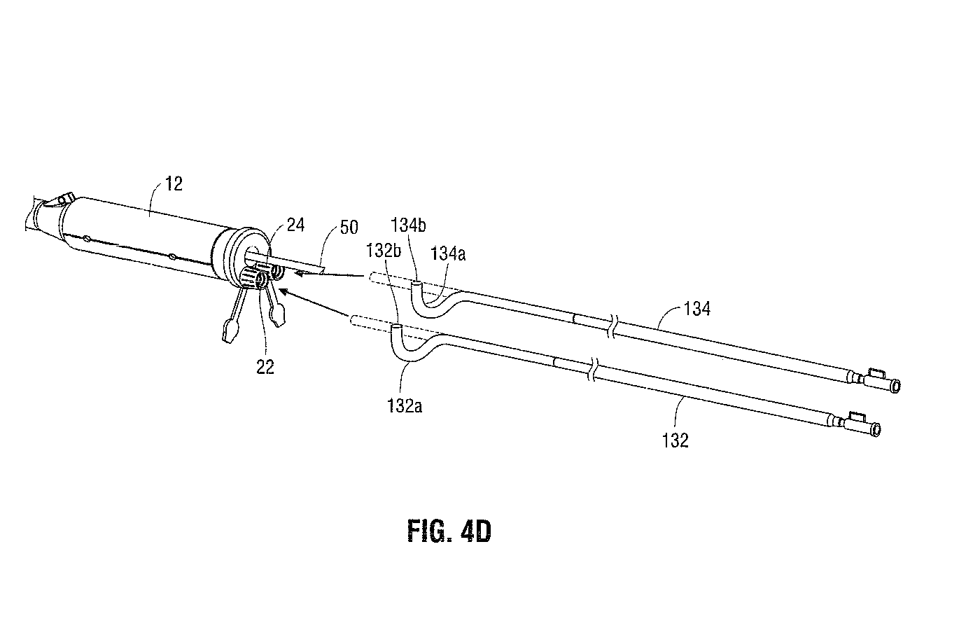

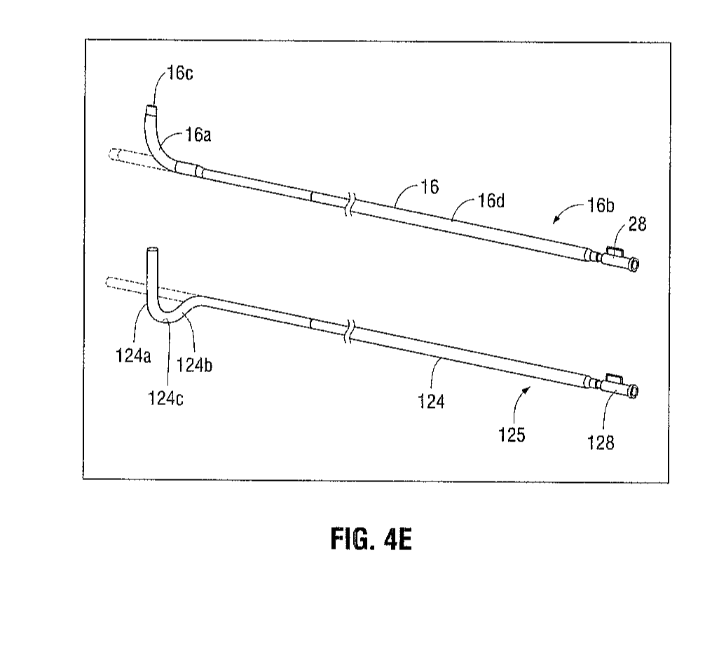

The tool channels 14, 16 are inserted through the proximal end 13 of the catheter 12 (see FIG. 4A) and advanced through respective lumens in the catheter 12. As shown in FIG. 4A, which illustrates a proximal portion 13 of catheter 12, the catheter 12 can include ports 22, 24 leading into the respective catheter lumens. The ports 22, 24 can include valves to maintain insufflation when the tool channels 14, 16 are inserted therethrough and translated axially therein. Tool channel (flexible tube) 14 has a pre-bent distal tip 14a providing a pre-curved distal end. Tool channel (flexible tube) 16 has a pre-bent distal tip 16a, providing a pre-curved distal end. In the embodiment of FIG. 4A, the two tool channels 14 and 16 are the same configuration and curvature. When the C-shaped tool channels 14, 16 are inserted into the lumens of catheter 12, the tips 14a, 16a are moved to a substantially straightened position substantially aligned with their longitudinal axis to facilitate advancement through the catheter lumens. When the tool channels 14, 16 are advanced sufficiently distally so the distal tips 14a, 16a are exposed from the confines of the walls of the respective catheter lumens, the tips 14a, 16a return to the pre-set curved position. This can be understood with reference to FIG. 4A which illustrates in phantom the straightened or linear position of the tool channels 14, 16 for movement within the catheter 12. As in the other embodiments disclosed herein, the tool channels 14, 16 can be composed of superelastic material, although other materials to provide the curved tip which returns from a substantially straight insertion shape when confined within the catheter to a curved shape when exposed from the catheter can also be used, such as stainless steel. Also, as in the other embodiments disclosed herein, shape memory properties of material such as Nitinol can be used with a memorized curved tip shape. The tool channels 14, 16 are unattached to the catheter 12 and the user can freely control, e.g., manipulate by hand, their axial movement from a proximal end portion 14b, 16b, which extends outside the catheter and the patient's body, during use.

The tool channels 14, 16 are removably insertable in the catheter 12. In this manner, tool channels 14, 16 can be exchanged for other tool channels during the surgical procedure. This is explained in more detail below.

The tool channels 14, 16 preferably have a larger diameter proximal region 14d, 16d, respectively. This larger outer diameter preferably is greater than an internal diameter of the respective lumens of the catheter (and/or the ports 18, 20), thus providing a stop to limit distal insertion of the tool channels 14, 16 so they do not fall inside the catheter 12.

The tool channels 14, 16 can optionally include markings 23, 25, (FIG. 1) respectively, at a region proximal to the catheter 12, i.e., exposed from the catheter 12, to provide a visual indicator to the user of the depth of insertion of the tool channels 14, 16 through the catheter lumens. That is, the markings can be utilized to help estimate the distance of the tip of the tool channels 14, 16 from the distal end of the catheter 12. The markings can also be provided on the enlarged proximal region 14d, 16d. The markings are shown in FIG. 1, it being understood that the markings can be provided in the other embodiments of the tool channels disclosed herein. The tool channels 14, 16 can have a luer fitting 26, 28, respectively, with a valve, at the proximal end which can close off backflow of insufflation gas from the body lumen, thus minimizing gas leakage between the tool channel and the instrument extending therein. This maintains insufflation when an endoscopic instrument (endoscopic tool) is inserted through the tool channels 14, 16 as described herein. The tool channels in an alternate embodiment shown in FIG. 6 have a hemostatic valve 30, 32 connected at a proximal end of tool channels 14′, 16′, respectively, to maintain insufflation during instrument insertion. As shown, valves 30, 32 are proximal of luer fittings 26′, 28′. The tool channels 14′, 16′ are identical to tool channels 14, 16 in all other respects. The tool channels can have an opening/access for gas insufflation into the body lumen such as for example in the side arm of valves 30, 32.

The tool channels (flexible guides) described herein can be color coded to improve the system's usability. For example, tool channel 14 can be of a first color, such as red, and tool channel 16 can be of a second color, such as black. Other colors can also be utilized. In this way, when the user is manipulating the flexible guides 14, 16 at their proximal ends outside the patient's body, the user will more readily see via the endoscope the corresponding color coordinated tip being manipulated within the body lumen. Note the entire flexible guide can have the same color or alternatively the matching color can be only at the proximal end visible to the user and the distal end visible by the endoscope. It should also be appreciated that instead of color coding, other indicia or markings can be provided so the user can match the exposed proximal end of the flexible tube with the distal end within the body lumen.

Markings can also be provided at the proximal region to indicate the direction of the pre-bent curve of the distal ends 14a, 16a.

The tool channels disclosed herein can in some embodiments be composed of a flexible soft material, such as Pebax. A superelastic nitinol backbone can in some embodiments be embedded in the wall of the Pebax material, e.g., within the curved portion. Other materials are also contemplated such as a single polymer layer, multiple polymer layers, a wire reinforced layer, or a combination thereof. In some embodiments, a tool channel can comprise (i) an inner layer of a polymer such as, for example TEFLON or polyethylene for slippery luminal surface on the inner diameter of the channel; (ii) a metal such as, for example, a stainless steel, nitinol, or cobalt chromium as a wire reinforcement in the configuration of a braid, mesh, or helical coil layer covering the inner layer; and, (iii) an outer layer of a polymer such as, for example, PEBAX, polyurethane, polyethylene, silicone, PVC, or nylon. Note the tool channels disclosed herein can in some embodiments be composed of a Pebax tubing, an overlying PVC tubing and polyolefin shrink tubing over the PVC tubing. This provides a balance between flexibility and rigidity, and also beefs up the proximal end to facilitate handling by the user.

The tool channels disclosed herein can be any size considered by one of skill to be useful in the systems described herein. For example, a tool channel can have an inner diameter ranging from about 1 mm to about 5 mm, from about 2 mm to about 4 mm, from about 1 mm to about 3 mm, or any range therein. The length of the tool channel complements the length of the system. For example, the tool channel can have a length ranging from about 40″ to about 72″, from about 48″ to about 60″, from about 42″ to about 70″, from about 44″ to about 68″, or any range therein.

In some embodiments, the tool channels can be configured such that the outer layer is (i) the most rigid in the proximal section of the channel (i.e., the first about 12″ to about 24″ of the channel), having a hardness of about 60 Shore D to about 80 Shore D; (ii) has a medium stiffness in the middle section (i.e., the next about 12″ to about 36″ of the channel), having a hardness of about 50 Shore D to about 72 Shore D; and, (iii) is the most flexible in the distal section (i.e., the next about 0.5″ to about 2″ of the channel), having a hardness of about 20 Shore D to about 50 Shore D). The distal section of the tool channel can in some embodiments be the section that flexes and can be the distal about 1″ of the channel. In some embodiments, the tool channels can have a rigid section just proximal to the distal section to keep this flexible section straight when there is a bending moment on the tip such as when the instrument which is inserted through the channel is grasping a tissue during a gastrointestinal treatment, for example. The length of the rigid section of the tool channels can range, for example, from about 1 cm to about 10 cm, from about 2 cm to about 8 cm, from about 3 cm to about 7 cm, from about 4 cm to about 6 cm, about 6 cm, or any range therein. The rigid section can include a rigid tube comprising a reinforcement material such as, for example, stainless steel or NITINOL, or a polymer such as PEEK or a polyimide embedded between the outer polymer layer and the inner polymer layer. The rigid section can have any suitable length to perform its function in the system. In some embodiments, the rigid section can have a length ranging from about 0.001″ to about 0.005″.

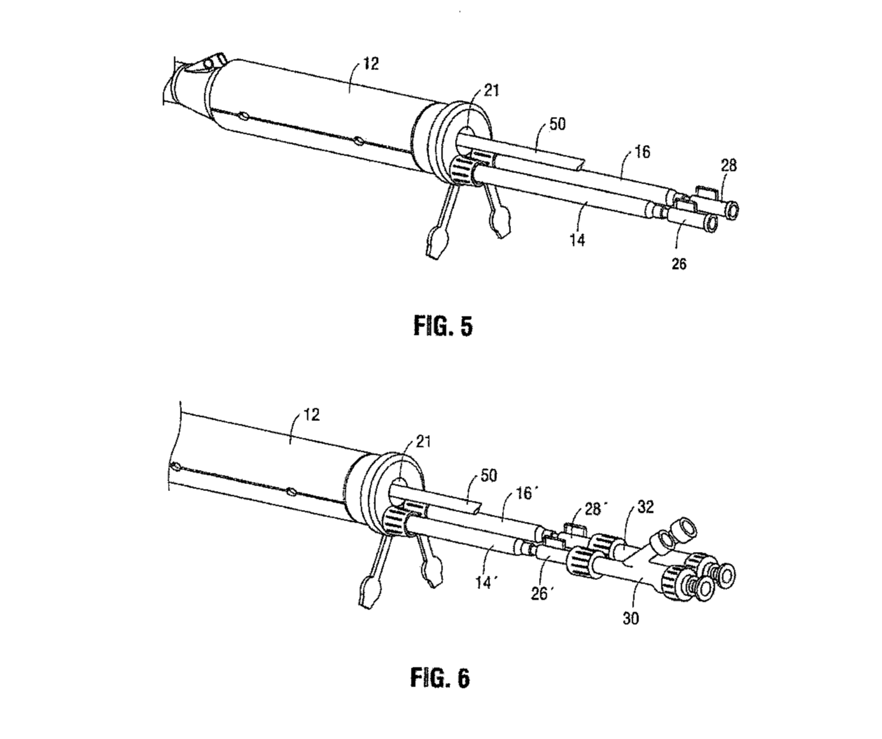

Catheter 12 also has a lumen 21 (see FIGS. 4A and 5) configured and dimensioned to receive an endoscope 50. In some embodiments, the lumen 21 is dimensioned to receive a conventional endoscope, e.g., a conventional colonoscope, and the catheter 12 is backloaded over the endoscope. This is described in more detail below in conjunction with the method of use. The lumen 21 can receive an articulating endoscope. In alternate embodiments, the endoscope can initially be inserted into the body lumen and the catheter inserted thereover.

With reference to FIG. 1 catheter 12 includes a handle (handle housing) 34 at the proximal portion 13. The handle can be any of a variety of shapes to provide a desired or ergonomic position for operation of the system. Catheter 12 also includes tubing 33 having a luer coupling and a control switch for closing off an internal gasket. Catheter 12 also has tubing 40 having a one-way stopcock to provide an insufflation port for providing gas insufflation into the body lumen. This port in some embodiments can be used to supplement the insufflation gas provided by the endoscope 50. The insufflation gas flows through lumen 21 in the area around the endoscope 50 since the cross-sectional dimension of the lumen 21 exceeds the cross-sectional dimension of the endoscope 50 to leave a sufficient gap. A seal can be provided within the housing anywhere along the handle. Seals can be provided in the catheter lumens to limit gas leakage in the space between the outer wall of the tool channels 12, 14 and the inner wall of the respective lumens of the catheter. Similarly, a seal can be provided in the lumen for the endoscope to limit gas leakage in the space between the outer wall of the endoscope and the inner wall of the lumen. Such seals within the lumens can be located in a region within the handle. In some embodiments, the seal is in the form of a membrane with a central round hole, and in some embodiments located distally in the handle 34 for the endoscope and proximally in the handle for the tool channels. This is shown for example in FIG. 18 where catheter 12′ has a handle 34′ with a membrane seal 42 for the endoscope within the endoscope lumen 21′, a membrane seal 44 for the lumen for tool channel 14 and a membrane seal 46 for the lumen for tool channel 16. Note other types of seals are also contemplated. In the illustrated embodiment, the endoscope seal 42 is in a distal region of the handle 34′ and the tool channel seals 44, 46 are in the proximal region of the handle 34′. Other locations are also possible including located in the same axial region or seal 42 proximal of one or both seals 44, 46. Note in some embodiments the seals can also provide some resistance to axial movement of the endoscope and axial movement of the tool channels to help retain their axial position until a predetermined force is applied. Catheter 12′ is identical to catheter 12 in all other respects.

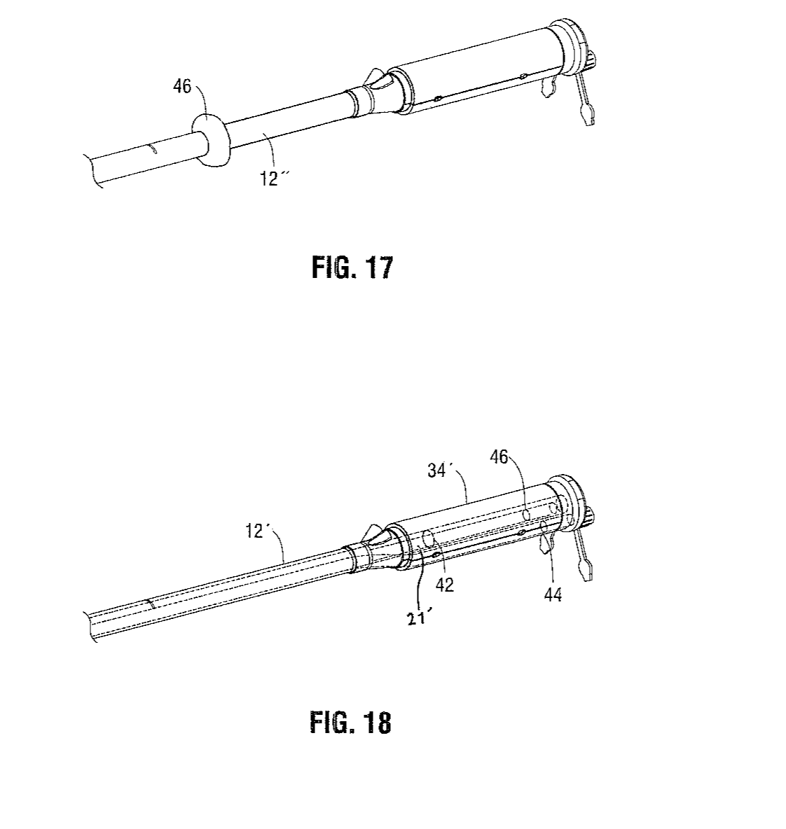

An external seal, such as a sponge-like cuff, balloon or other blocking device, can be provided such as cuff 46 shown in FIG. 17. Cuff 46 is positioned around the catheter shaft 12″, preferably at a proximal portion, to occlude the space, e.g., rectal space, between the catheter shaft 12″ and the body space wall, e.g., rectal wall, to minimize leakage. Catheter 12″ is identical to catheter 12 in all other respects or to catheter 12′ if provided with internal seals.

Since the catheter 12 does not have a retractor system or stabilizer, the need for sliders or other actuators is eliminated and the handle forms a receptacle for the endoscope and the tool channels (flexible guides). Thus, the catheter can be viewed as a flexible single port for instrument insertion for endoscopic surgery such as in the manner of current single ports used for laparoscopy.

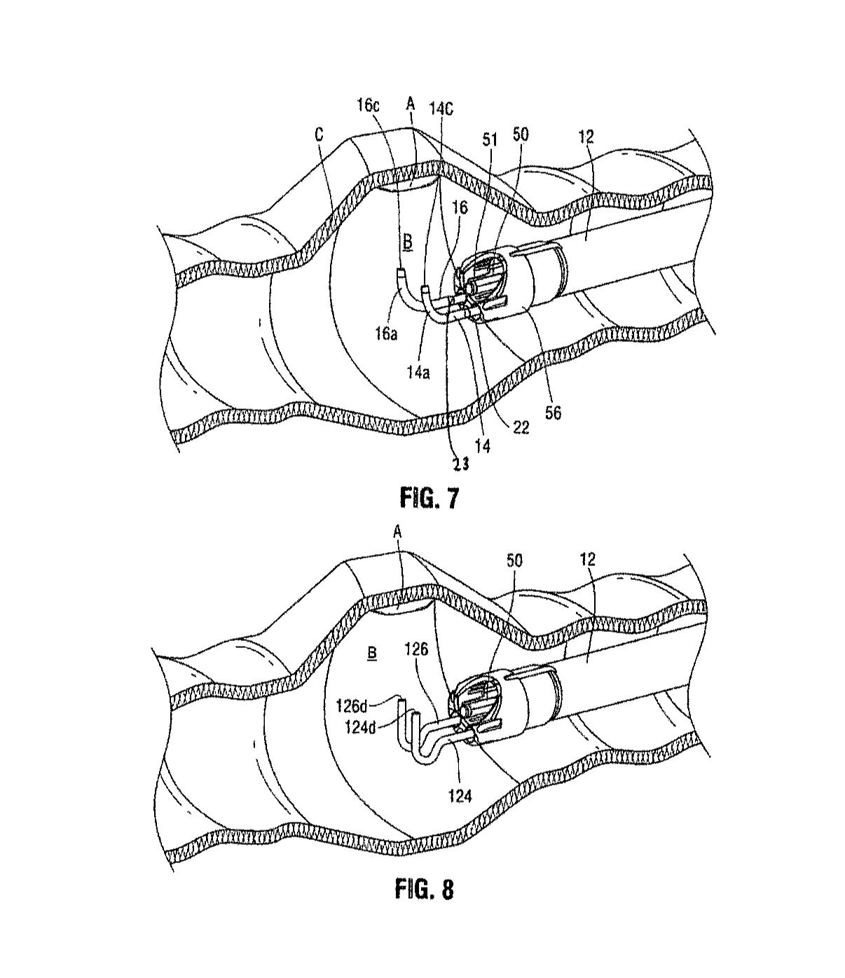

At the distalmost end of the catheter 12 are distal openings 22, 23 (FIG. 7) which form distal openings for the lumens receiving the tool channels 14, 16. Thus, the catheter 12 terminates at the distal openings 22, 23 which are substantially axially aligned with the distalmost edge of the catheter. The tool channels 14, 16 extend distally past the distal openings 22, 23 to extend into the insufflated expanded space in the body lumen for guiding endoscopic instruments for performing the surgical procedure within the body lumen. The catheter 12 can optionally include a cap 56 (FIG. 7) at its distal end or alternatively the distal end can be a smooth continuation of the outer wall. Lumen 21 for receiving the endoscope terminates in a distal opening substantially aligned with the distalmost edge of the catheter 12 so the endoscope can provide visualization distal of the distal end of the catheter. As shown, the distal openings 21, 22, and 23 in a preferred embodiment terminate at a substantially axially aligned position which is at the terminal end (distalmost edge) of the catheter 12. The system relies on insufflation to expand the body lumen (space) distal of the distalmost edge of the catheter 12.

In some embodiments, the wall of the catheter 12 can be wire-reinforced, such as mesh, braided, or the like, to provide kink resistance and torqueability to the system, as well as to further facilitate a positioning of the system in the subject.

The system 10 of FIG. 1 is shown in FIG. 7 for use in removing a lesion A, such as a polyp, from a colon wall C of colon B, it being understood, however, that the system 10 can be used for other procedures within the colon or the gastrointestinal tract, as well as used for other procedures in other body lumens or body spaces of a patient such as in the biliary tree, bronchi, trachea, uterus, ovarian tube, vessels, etc. Note the colon (or other body space/lumen) is preferably insufflated to expand the space as shown in FIG. 7 to provide an increased area for manipulation of endoscopic tools and to enhance visualization.

Distal viewing endoscope 50, in which the catheter 12 has been advanced over the proximal end, or alternatively backloaded over the distal end, so the endoscope extends through lumen 21, is inserted through the lumen in the colon B in a procedure to remove the target tissue, e.g., polyp A, from the wall C of the colon B. The endoscope 50 in this embodiment is a distal viewing scope with a wide distal viewing area of for example about 150 to about 170 degree range so the polyp A and surrounding area can be visualized. After placement of the scope 50 adjacent the target issue, e.g., slightly proximal of the target polyp C, the catheter 12 is further advanced over the endoscope 50 until it reaches the target site (compare FIGS. 2B and 3). As shown in FIG. 7, in this position, the distal end 51 of the endoscope 50 is preferably positioned at the end of cap 56 and does not extend into, or significantly into, the working space within the colon B to thereby leave more room for maneuvering of the endoscopic instruments within the working space. Other positions, however, are also contemplated, e.g., in some versions the endoscope can extend into the working space.

Next, tool channels 14, 16 are inserted through the entry ports 22, 24 in the proximal region 13 of the catheter 12 and advanced by the user through the catheter lumens so they extend out the distal openings 22, 23 of the respective lumens and into the insufflated expanded working space. Seals such as those described above can limit gas leakage. Note as they emerge from the lumens 22, 23 and out of the confines of the walls of the lumens of the catheter 12, their distal tips 14a, 16a return to their pre-curved (bent) position, curving upwardly (as viewed in the orientation of FIG. 7) toward the polyp A, with their distal openings 14c, 16c facing, or rotated to face via rotation of the tool channels 14, 16, in the same direction toward the polyp A. Note the tool channels 14, 16 can be independently rotated and/or moved axially to adjust their position with respect to the polyp A. As can be appreciated, the terms upwardly and downwardly as used herein refer to the orientation of the system in the referenced Figures. If the position of the system changes, the orientation and terms would also change.

After insertion of the tool channels 14, 16, endoscopic instrument (tool) 62 is inserted through the luer fitting 26 of the tool channel 14 and advanced axially through the lumen (channel) of the tool channel 14, exiting the distal opening 14c in the tool channel 14. As shown in FIG. 9A, a first endoscopic instrument 62 extends from tool channel 14 and follows the curve of the tool channel 14. A second endoscopic instrument (tool) 64 is inserted through the luer fitting 28 of tool channel 16 and advanced through the lumen of the tool channel 16, exiting the distal opening 16c in the tool channel 16. As shown in FIG. 9A, the second endoscopic instrument 64 extends from the tool channel 16 and follows the curve of the tool channel 16. As noted above, the tool channels 14, 16 can include a seal or valve, such as the hemostatic valves as shown in FIG. 6, so insufflation is not lost during insertion and removal of the endoscopic instruments 62, 64 from the tool channels 14, 16. The endoscopic instruments 62, 64 can be moved further axially as shown in FIG. 9B to extend further from the tool channels 14, 16 to contact and treat, e.g., remove, the polyp A. This movement of the endoscopic instruments and the advantage of the tool channels 14, 16 are shown by comparing FIGS. 9A and 9B. As can be seen, once the tool channels 14, 16 are in the desired position with respect to the polyp A, they can be considered as defining a fixed curve. This means that when the endoscopic instruments 62, 64 are axially advanced, they move closer to the target polyp A, without a change in curvature and without a change in their axial position with respect to the polyp A, thus providing an extra degree of freedom. The endoscopic instrument 62, which in the illustrated embodiment is a grasper, applies tension on the polyp A while the endoscopic instrument 64 which in the illustrated embodiment is in the form of an electrosurgical dissector dissects/severs the polyp A from the colon wall C. Other endoscopic instruments for polyp removal can also be utilized. As can be appreciated, in this manner, the endoscopic instruments are separate from the endoscope as compared to inserting the instruments through the working channel of the endoscope, thereby providing movement independent of endoscope movement. Such approach resembles laparoscopic surgery in which the visualization device is separate from the working instruments. Note, in some embodiments, a single tool channel can be utilized and another endoscopic instrument, e.g., a grasper or a dissector, can be inserted through a working channel (lumen) of the endoscope. Such instrumentation inserted through an endoscope can also be utilized with the embodiments having two or more tool channels.

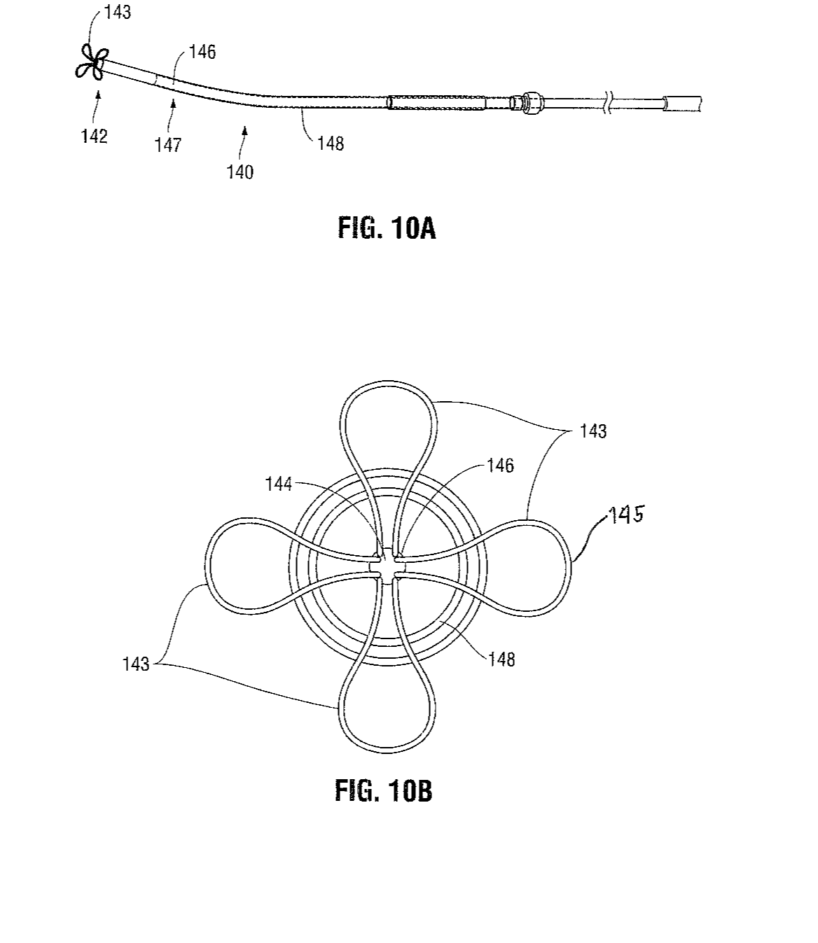

FIGS. 10A and 10B illustrate another endoscopic instrument that can be inserted through the tool channel. The instrument 140 is a tissue retractor configured to gently but firmly push bulky tissue, such as a large polyp, away from the dissection plane (operating field) within the body lumen, e.g., the colon, during the surgical procedure. The tissue retractor is configured to retract bulky tissue which would otherwise obscure the dissection plane during the procedure and might not be able to be sufficiently moved by the jaws of conventional graspers. For example, in certain instances, a large polyp could occupy ½ or even ⅔ of the body lumen, and during the procedure becomes floppier, moving in different directions to obstruct the view of the dissecting plane. The tissue retractor provides loops or petals which have sufficient rigidity to move the polyp (or other tissue) and provide a relatively large interface between the loops and the polyp (or other tissue) to enhance retraction. The pre-bent tips of the tool channels bend the tissue retractor toward the target tissue. More specifically, the tissue retractor 140 has a distal portion 142 with a distal opening 144 and a proximal portion. An inner shaft (inner member) 146 is slidably positioned within outer tube or sheath (outer member) 148, preferably having a curvature as shown so a distal region 147 is at an angle to the longitudinal axis. At the distal end of inner shaft 146 is attached a plurality of closed loops or petals 143. Although four petals (loops) 143 are shown, it should be appreciated that a fewer number or a greater number of petals 143 could be provided. The petals 143 are movable from a collapsed insertion position within the outer tube 148 to an advanced position where they are exposed from the outer tube 148 and can move to the expanded position. The petals 143 are exposed by advancement of the inner shaft 146 relative to the outer tube 148. However, it should be appreciated, that alternatively, the outer tube could be retracted to expose the petals 143 or both the inner shaft could be moved proximally and the outer tube moved distally. In each of these methods, it is the relative movement of the inner shaft and outer tube that exposes the petals 143 for expansion.

The tissue retractor 140 can include a locking mechanism to retain the inner shaft 146 in the desired axial position to thereby retain the retractor petals (loops) 143 in the desired expanded position. Further details of the tissue retractor and locking mechanism are disclosed in application Ser. No. 15/148,999, filed May 6, 2016, the entire contents of which are incorporated herein by reference.

Petals 143 are shown in the illustrated embodiment as closed loops. The petals 143 can be formed from wires attached to the inner shaft 146. The petals 143 can be made of shape memory material with a memorized configuration of FIGS. 10A and 10B so that when exposed from the outer tube 148 they are no longer constrained and return to their expanded memorized position. In some embodiments, the petals 143 are configured to “spring out” once partially exposed. That is, once a sufficient area of the petals 143 is exposed from the outer tube 148, they “spring out” to their expanded position.

In the illustrated embodiments, the petals 143 have a curved outer surface 145 which can be angled with respect to a longitudinal axis of the inner shaft 146. That is, an axis extending through the apex of the loop can be at an angle to the longitudinal axis of the inner shaft 146 so that one or more of the loops 143 are at an angle to the longitudinal axis. Alternatively, the loops can be substantially perpendicular to the longitudinal axis. The loops 143 have sufficiently flexibility to be collapsible for insertion while having sufficient rigidity to move/retract tissue. Various portions of the loops can be utilized to contact and move tissue. The four loops illustrated are the substantially the same size, however, in alternate embodiments, loops of different sizes could be provided.

The loops (petals) 143 in some embodiments are covered with a thin pliable material, such as a plastic, cloth or other materials, to provide a barrier to prevent tissue protruding into (through) the loops. In another alternate embodiment, a thin pliable material covers two of the loops 143 and a thin pliable material covers another two of the loops 143. This material can be a plastic, cloth or other materials. Materials each provide a barrier for protrusion of the tissue into (through) the loops as well as to prevent tissue protruding between the two covered loops. It should also be appreciated that in alternate embodiments, the pliable material described herein can cover more than two of the loops or all of the loops. Additionally, alternatively, the pliable material can be placed between adjacent loops and not over the loops. Thus, various combinations of intra-loop and inter-loop coverings can be provided.

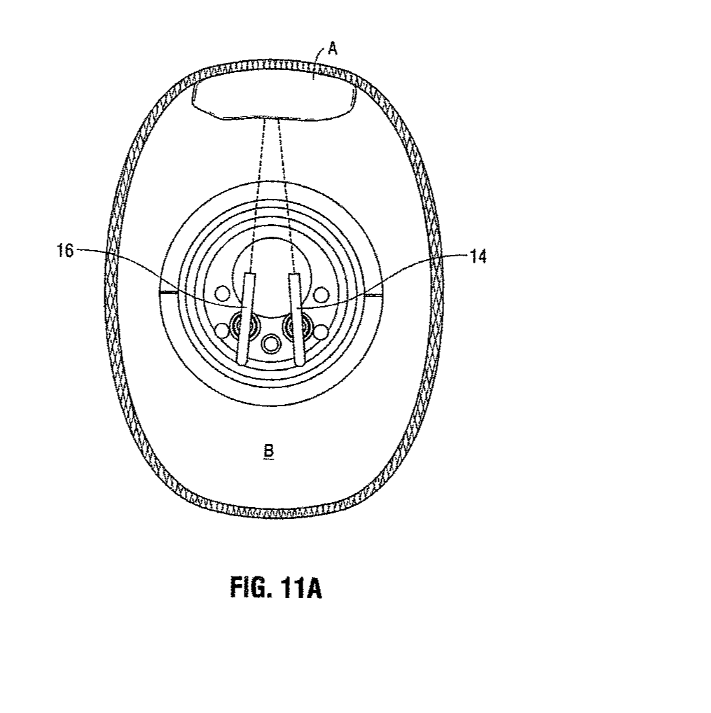

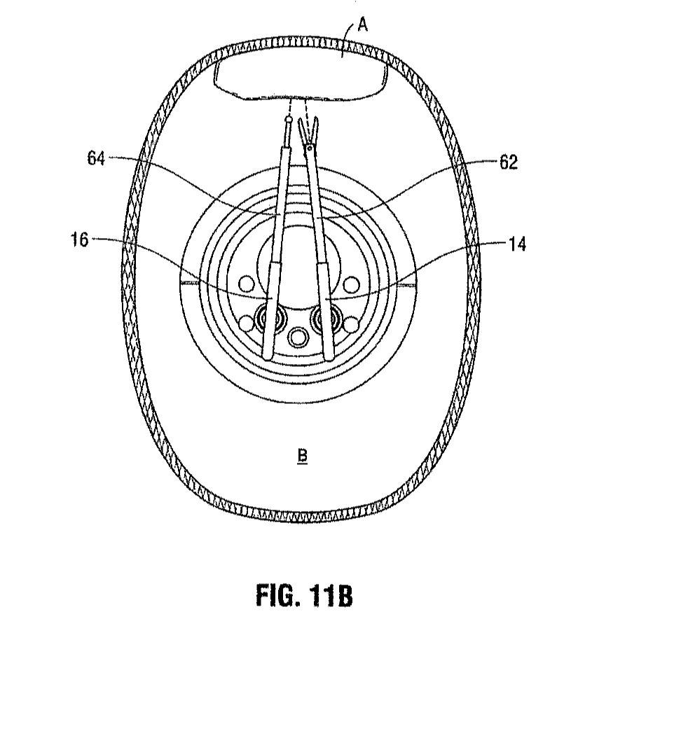

Due to the angles of the tool channels 14, 16 and thus the endoscopic instruments inserted therethrough, tissue triangulation can be achieved as depicted by the dotted lines in FIG. 11A which shows the path from the tool channels 14, 16 to the target tissue, e.g., polyp A. FIG. 11B shows such triangulation with the instruments 62, 64 extending from the tool channels 14, 16.

Note that the tool channels enable in some embodiments the use of current instruments, e.g., off the shelf instruments, since change of angle (articulation) can be achieved by the tool channels (flexible guides) without the instruments having an articulation mechanism.

In some embodiments, the tool channels are held within the catheter and/or the endoscopic instruments are held within the tool channels by frictional resistance. This is achieved by the seal within the catheter lumen providing some resistance to tool channel movement within the catheter lumen and the seal within the lumen of the tool channels providing some resistance to movement of the flexible instrument within the tool channel lumen. Also, since the tool channels and instruments are not straight, this provides additional resistance to movement. An instrument being advanced distally through the C or S-curve of the tool channel will encounter resistance to help keep the instrument in place. Thus, the resistance between the flexible instrument and the curved tool channel creates a functional “lock” which in certain embodiments would need to be overcome with lubrication and/or sufficient manual force by the user. Such functional lock can also occur in some embodiments by the resistance of the tool channel within the catheter lumen requiring sufficient manual force and/or lubrication.

After removal of the polyp A from the colon wall C, the polyp is removed from the body. Catheter 12 is then removed from the colon B.

In some embodiments, for one of the tool channels, instead of it having a lumen extending therethrough, it could be solid with a thread at the tip. A clip is then delivered through a working channel of the endoscope or through another tool channel to fix the thread to the lesion.

FIG. 4B illustrates an alternate embodiment of the tool channels. In this embodiment, tool channels 114, 116 are identical to tool channels 14, 16 of the FIG. 4A embodiment except for the curvature of the distal end. More particularly, tool channels 114, 116 have a pre-bent (pre-curved) C-shaped distal end 114a, 116a, respectively, to provide a pre-curved distal end, with a radius of curvature greater than the radius of curvature of tool channels 14, 16. When the tool channels 114, 116 are inserted into the lumens of catheter 12, the tips 114a, 116a are preferably substantially straightened to facilitate advancement through the lumens (see phantom lines) in the same manner as tool channels 114, 116. When the tool channels 114, 116, are advanced sufficiently distally so the distal tips 114a, 116a are exposed from the confines of the walls of the catheter lumens, the tips 114a, 116a return to the pre-set curved position in the same manner as tool channels 14, 16 described above. In certain surgical procedures, or during the procedure, the clinician might desire a different curvature to be utilized, e.g., the tool channels 114, 116 will have a less sharp curve (longer transition) within the body lumen than tool channels 14, 16, tool channels 14, 16 will take up less longitudinal space within the body lumen than tool channels 114, 116, tool channels 14, 16 have a more abrupt lateral change of direction than tool channels 114, 116, etc. Note FIG. 4B illustrates one variation of the curvature, it being understood that other curvatures are also contemplated.

FIG. 4C illustrates another alternate embodiment of the tool channels. In this embodiment, tool channels (flexible tubes or guides) 124, 126 are identical to tool channels 14, 16 of the FIG. 4A embodiment except for the curvature of the distal end. More specifically, tool channel 124 has a double curve (bend) at its distal tip 124a defining a first curve (bend) 124b extending away (downwardly as viewed in the orientation of FIG. 4C) from the longitudinal axis and then transitioning into a second curve 124c extending in a second opposite direction (upwardly as viewed in the orientation of FIG. 4C) toward the longitudinal axis. Tool channel 126 similarly has a double curve (bend) at its distal tip 126a defining a first curve (bend) 126b extending away (downwardly as viewed in the orientation of FIG. 4C) away from the longitudinal axis and then transmitting into a second curve 126c extending in a second opposite direction (upwardly as viewed in the orientation of FIG. 4C) toward the longitudinal axis. Thus, the distal tips 124a, 126a form a somewhat S-shaped curve. The first curve increases the distance from the distal opening 124d, 126d of the tool channel 124, 126, respectively, to the target lesion as compared to a single curve which does not have a downward bend. The tool channels 124, 126, like the other tool channels disclosed herein, are inserted through the proximal end of the catheter 12 (through ports 22, 24) and advanced through respective lumens in the catheter 12. As described above, ports 22, 24, communicate with the catheter lumens and can include valves to maintain insufflation when the tool channels 124, 126 (or other tool channels disclosed herein) are inserted therethrough and translated axially therein.

When the tool channels 124, 126 are inserted into the lumens of catheter 12, the pre-bent tips 124a, 126a are preferably substantially straightened (substantially aligned with their longitudinal axis) to facilitate advancement through the lumens. When the tool channels 124, 126 are advanced sufficiently distally so the distal tips 124a, 126a are exposed from the confines of the walls of the catheter lumens, the tips 124a, 126a, return to the pre-set double curved position. This can be understood with reference to FIG. 4C which illustrates in phantom the straightened position of the tool channels 124, 126 for movement within the catheter 12. As in the other embodiments disclosed herein, the tool channels 124, 126 can be composed of superelastic material, although other materials to provide the curved tip which returns from a substantially straight insertion shape to a curved shape when exposed can also be used, such as stainless steel. Also, as in the other embodiments disclosed herein, shape memory properties of material such as Nitinol can be used with a memorized curved tip shape. The tool channels 124, 126, like the other tool channels disclosed herein, are preferably unattached to the catheter 12 so that the user can freely control their axial movement from a proximal end portion 125, 127, respectively during use.

The tool channels 124, 126 can optionally include markings like markings 23, 25 of the embodiment of FIG. 1 at a region proximal to the catheter 12 to provide a visual indicator to the user of the depth of insertion of the tool channels 124, 126 through the catheter lumens. The tool channels 124, 126 can also be color coded or provided with some other indicator for visual differentiation as described above with respect to tool channels 14, 16. The tool channels 124, 126 can have a luer fitting 128, 129, respectively, (with a valve), at the proximal end which can close off backflow of insufflation gas from the body. This maintains insufflation when the endoscopic tool is inserted through the tool channels 124, 126. The tool channels in an alternate embodiment can have a hemostatic valve like valves 30 and 32 of FIG. 6 to maintain insufflation during tool insertion.

FIG. 8 is a view similar to FIG. 7 except showing use of the tool channels 124, 126 within a colon B by way of example. The tool channels 124, 126 are shown extending beyond the distal edge of catheter 12 out the distal openings and into the working space expanded by insufflation.

In the embodiment of FIG. 4C, the distal tip of the tool channels 124, 126 extend radially beyond the longitudinal axis of the tool channel 124, 126 so the distal opening is beyond the axis. In the alternate embodiment of FIG. 4D, the double curved (somewhat S-shape) distal tip 132a, 134a of tool channels (flexible guides) 132, 134 do not extend radially beyond the longitudinal axis so the distal opening 132b, 134b is substantially aligned with the longitudinal axis of the respective tool channels 132, 134. This reduced length after (distal) the second curve increases the distance from the distal opening to the lesion. The tool channels of FIG. 4D are otherwise identical to the tool channels of FIG. 4C. Alternatively, the distal opening of the tool channels could be below (as viewed in the orientation of FIG. 4D) the longitudinal axis of the tool channels to further increase the distance from the distal opening to the target lesion.

The various shaped/configured removably insertable tool channels described herein enable exchange during a surgical procedure. Different tool channels can be provided in a kit, such as, by way of example, the kit shown in FIG. 4E, having a tool channel 16 and a tool channel 124. Other kits containing different combinations of the tool channels disclosed herein can be provided. Although two tool channels are shown in the kit of FIG. 4E, more than two tool channels can be provided in the kit and various combinations of tool channels can be provided in the kit. Thus, any combinations and any number of tool channels can be provided in separate kits. It is also contemplated that identical tool channels be packaged in the same kit. In any event, the various tool channels enable the clinician to initially select as well as subsequently exchange the tool channels during the surgical procedure if a larger or smaller curvature is desirable or an increased distance to the target tissue is desirable such as that provided by the double curved tips, etc. Consequently, the clinician can decide after viewing the body space via the endoscope which tool channels are best suited. Due to their removability, at any time during the procedure, the tool channel can be removed and replaced (exchanged) for another tool channel having a different configuration, e.g., a different radius of curvature or a different curved shape, e.g. single curve or double curve.

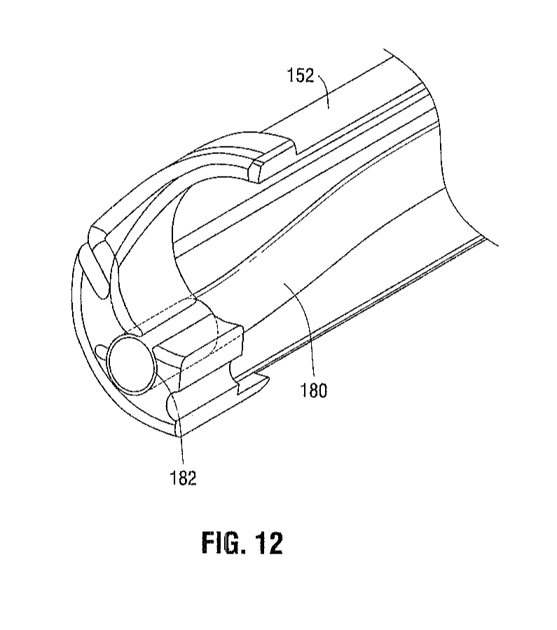

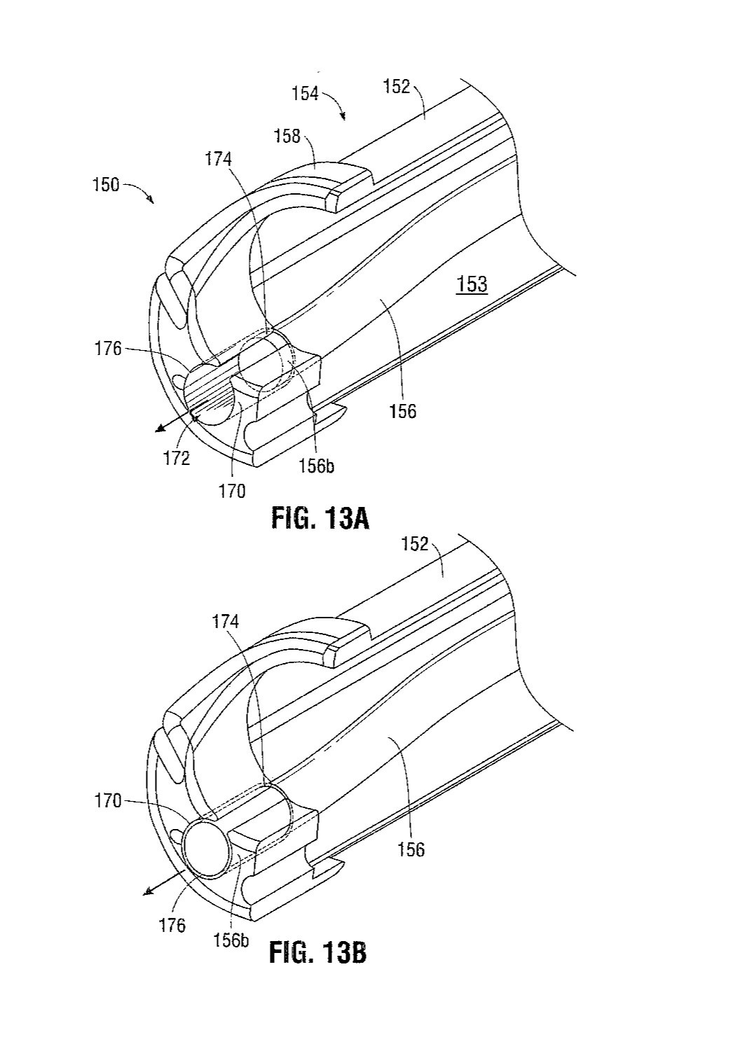

FIGS. 12-16 illustrate alternative embodiments of the system of the present invention. The system includes floating (flexible) channels within the outer tube. In one embodiment, the floating channels are fixed at their proximal and distal ends as in FIG. 12; in another embodiment, the floating channels are fixed at their proximal ends but are unattached at their distal ends as in FIGS. 13A-16. As can be appreciated from the discussion below, the floating channels reduce the overall stiffness of the catheter (outer tube) which would otherwise be stiffer if the channels were fixed along their entire length and did not float within the catheter. The floating channels also reduce kinking of the tool channels (flexible guides) inserted through the floating channels and reduce kinking of the instruments (tools) inserted through the tool channels.

Turning to the embodiment shown in FIGS. 13A-16, the system 150 includes a flexible catheter or outer tubular member (main tube) 152. The distal portion of the outer tube 152 is illustrated and designated generally by reference numeral 154. The handle housing (not shown) at the proximal portion is similar to the previously described handle housing, having access ports for inflow tubes (not shown) which can be part of a member (organizer) secured within the handle housing, an opening for entry of an endoscope into the outer tube 152, ports to provide entry for the tool channels (flexible guides) which preferably include a valve to maintain insufflation when the tool channels are inserted and translated therethrough, seals, etc.

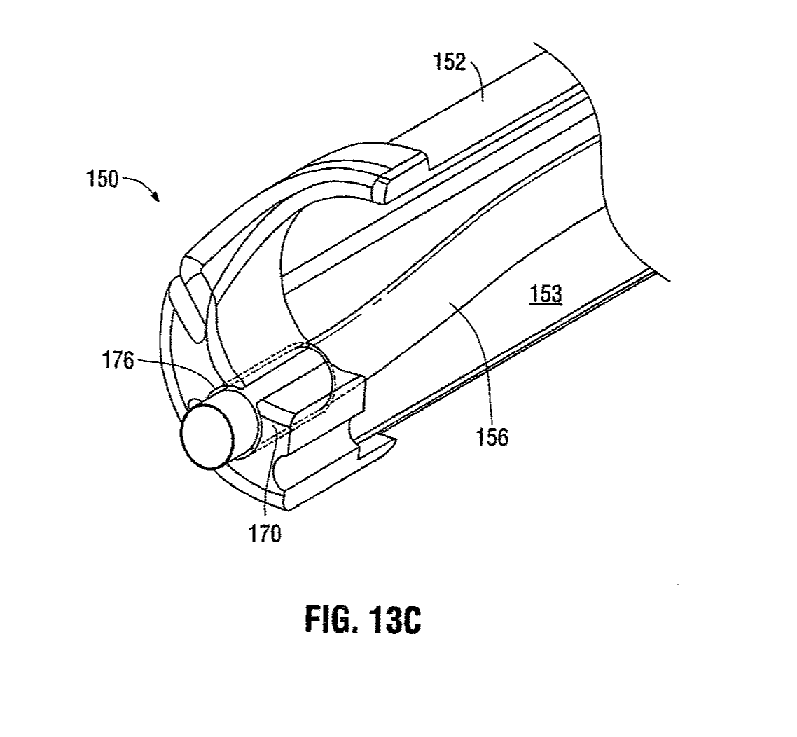

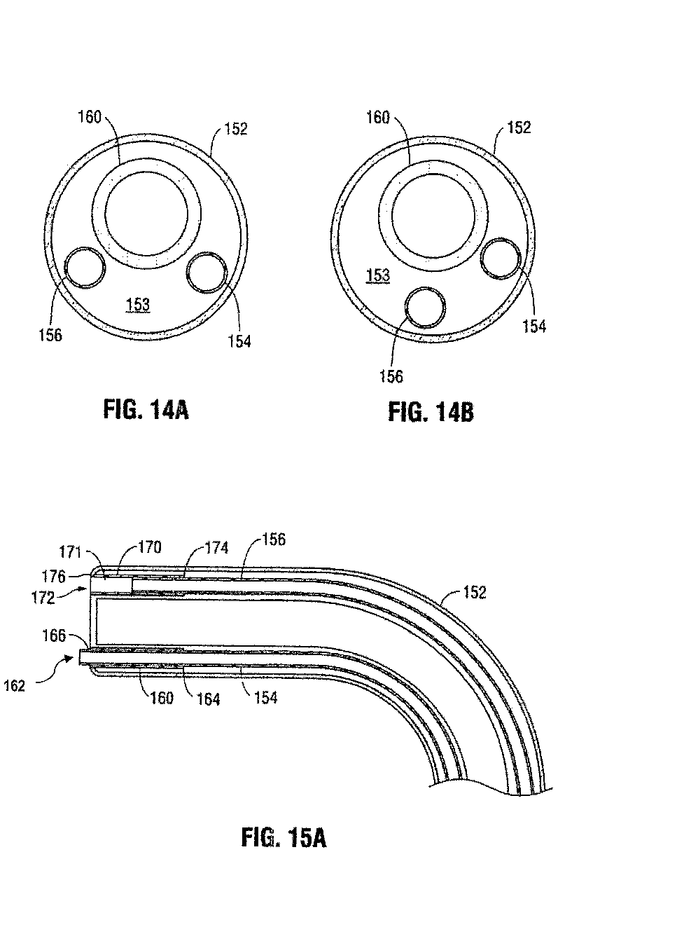

With reference to the cross-sectional view of FIG. 14A, the outer tube (catheter) 152 in this embodiment has a single lumen 153. This lumen 153 is dimensioned to receive 1) an endoscope, such as the endoscopes described above; and 2) two flexible floating channels 154, 156 each configured to receive a tool channel. For clarity, only one flexible channel, channel 154, is shown in FIGS. 13A-13C. As noted above, the terms flexible guides and tool channels are used throughout this application interchangeably. The two flexible channels 154, 156 are in the form of flexible tubes and float inside the lumen 153. That is, the two floating channels 154, 156 have intermediate portions that can move radially (laterally) within the lumen 153 of the outer tube 152. Stated another way, the floating channels 154, 156 are unconstrained within the outer tube 152 so they can bend relative to the outer tube 152 so their bending action does not need to follow that of the outer tube 152. In this manner, when the outer tube 152 is inserted in the body lumen and needs to bend to accommodate the curvatures of the body lumen, e.g., the gastrointestinal tract, the flexibility of the outer tube 152 is maintained since the floating channels 154, 156 can move within the lumen 153. As can be appreciated, if the two channels were fixed with respect to the outer tube 152 so there was no bending or movement with respect to the outer tube 152, and the channels were forced to bend in conformity with the outer tube 152, the outer tube 152 would be much stiffer as the channels would have to carry the bending stresses which could limit bending of the catheter and/or cause kinking of the tool channels or tools extending through the tool channels within the catheter. Thus, in the embodiments of the present invention which include the floating channels, these advantages of increased flexibility are achieved. It should be understood that any of the systems disclosed herein could be provided with floating channels. Likewise, any of the systems disclosed herein could be provided without floating channels. FIG. 14B provides by way of example a location of the floating channels 154, 156 when they are moved within the catheter 152 as it is bent. Clearly, floating channels 154, 156 will move to various other positions in response to catheter bending. FIGS. 13A and 13B show a cutaway view of the distal end of outer tube 152 illustrating one of the floating channels for ease of illustration.

Also, by providing a single lumen in this embodiment to receive the endoscope and the tool channels, rather than separate lumens which would require additional wall structure, a smaller diameter catheter can be provided which also reduces the overall stiffness of the catheter.

The endoscope 160 in the embodiment of FIG. 14A also floats within the lumen 153. That is, the endoscope 160 occupies only a certain region of the lumen 153 and can move radially (laterally) within the lumen 153 of outer tube 152 to increase the flexibility of the system. Thus, the endoscope 160 can move relative to the outer tube 152 in a similar manner as the floating channels 154, 156 can move relative to the outer tube 152. In alternate embodiments, a floating channel can be provided for the endoscope. The endoscope in some embodiments can be allowed to float in a channel that is substantially larger than the endoscope, providing a sliding motion for the endoscope as well as room for side-to-side movements as well.

In one embodiment by way of example, the internal diameter of the lumen 153 of the outer tube 152 can range between about 5 mm and about 50 mm and is preferably about 10 mm to about 20 mm. Each of the floating channels can preferably have an outer diameter of about 2 mm to about 10 mm, and preferably about 5 mm. The endoscope typically has a diameter of about 2 mm to about 20 mm and is preferably about 5 mm to about 12 mm. Thus, as can be appreciated, the floating channels and endoscope occupy only a percentage of the internal lumen 153, leaving sufficient room for movement. Note that other dimensions and thus ratios of the floating channels and endoscope to the internal diameter of the lumen 153 are also contemplated for the systems disclosed herein.

In one embodiment, by way of example, the outer tube 152 has a length of about 10 cm to about 200 cm, and more preferably about 60 cm to about 90 cm. The floating channels 154, 156 can have a length of about 10.1 cm to about 204 cm, and preferably about 60.5 cm to about 91 cm, thereby exceeding the length of the outer tube 152. Other dimensions are also contemplated. This greater length of the floating channels 154, 156 in the embodiments where they are fixed at both the proximal and distal ends enables the floating movement.

Floating channel 154, also referred to herein as a first flexible channel or a first floating channel or a first flexible tube, has a proximal end 154a and an opposing distal end 154b. Channel 156, also referred to herein as a second flexible channel or a second floating channel or a second flexible tube, has a proximal end 156a and an opposing distal end 156b. Note the terms “first” and “second” to describe various components of the systems of the present disclosure are used herein for ease of description. Note in the embodiments of FIGS. 12-16 two floating channels are provided. It is also contemplated that only one floating channel is provided or more than two floating channels are provided.

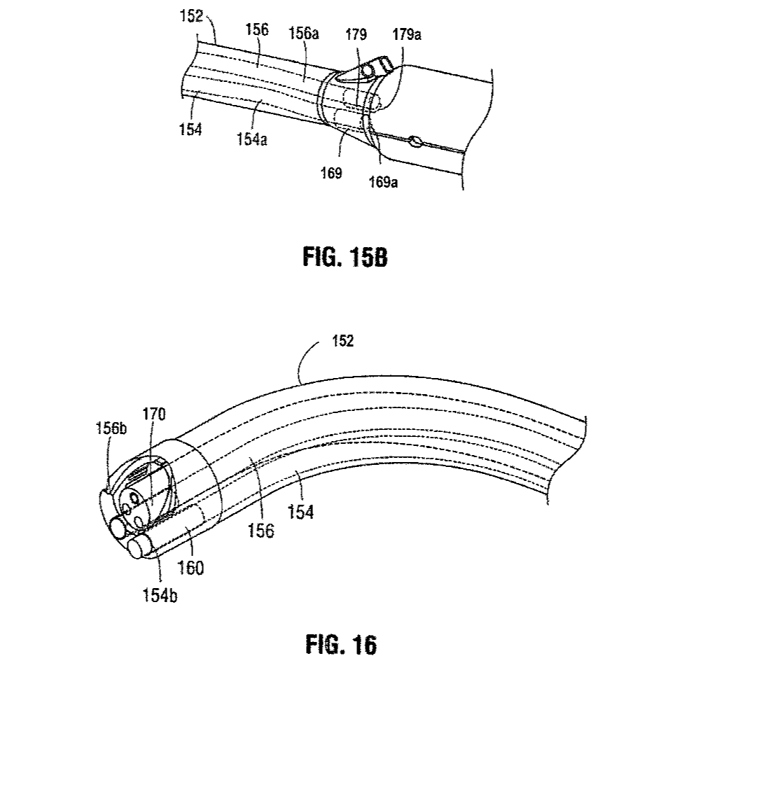

Positioned with the outer tube 152 at a distal end is a first fixed distal tube 160 which forms a pocket for the first floating channel 154. First distal tube 160 has an opening 162, a proximal edge 164 and a distal edge 166. In some embodiments, instead of an opening 162 the distal end can be closed. Preferably, distal edge 166 is substantially flush with the distal edge of the catheter 152 or end cap 158 if provided. At the proximal end of the system, positioned either within the outer tube 152 or alternatively at a distal region of the handle housing, is a first fixed proximal tube 169 having a proximal edge 169a, as shown in FIG. 15b which illustrates the proximal end of the floating channels 154, 156.

Also positioned with the outer tube 152 at a distal end is a second fixed distal tube 170 which forms a pocket for the second floating channel 156. Distal tube 170 has an opening 172, a proximal edge 174 and a distal edge 176. In some embodiments, instead of an opening 172 the distal end can be closed. Preferably, distal edge 176 is substantially flush with the distal edge of the catheter 152 or end cap 158 if provided. At the proximal end of the system, positioned either within the outer tube 152 or alternatively at a distal region of the handle housing is a second fixed proximal tube 179 having a proximal edge 179a. The first and second proximal tubes 169, 179 are preferably attached to an inner wall of the outer tube 152 or handle housing by bonding or welding or other attachment methods. Similarly, the first and second distal tubes 160, 170 are preferably attached to an inner wall of the outer tube 152 by bonding or welding or other attachment methods. Note in FIG. 15B, the fixed proximal tubes 169, 179 are shown cutaway (into a half cylinder) for clarity, it being understood that the tubes can be cylindrical in configuration like the distal fixed tubes 160, 170. Other configurations for the fixed distal and proximal tubes are contemplated.

The floating (flexible) channels are fixed at their proximal end but remain free (unattached) at their distal ends. More specifically, FIGS. 13A-13C illustrate a cutaway view of the system so that only one of the floating channels, the second floating channel 156, is illustrated. The first floating channel 154 is attached and configured in a similar fashion as second floating channel 156. Second floating channel 156 is attached at its proximal end in the same manner as floating channel 154, i.e., attached within a fixed proximal tube 179. The first floating channel 154 is not shown in FIGS. 13A-13C but is shown in FIGS. 15A-15B and 16. The floating channels 154, 156 are unattached at their distal ends to form telescoping channels within the outer tube (or catheter) 152 and attached at their proximal end, i.e., attached within the fixed proximal tube 169, 179. Note in the alternate embodiment of FIG. 12, the floating channels differ from the floating channels 154, 156 of FIG. 13A in that they are attached at their distal ends as well as their proximal end as discussed below.

More specifically, with continued reference to FIGS. 13A-13C and FIG. 15A, the first fixed distal tube 160 is attached within the outer tube 152 adjacent optional end cap 158 positioned over the outer tube (catheter) 152 of the system 150. First fixed distal tube 160 forms a pocket for the first floating channel 154. Distal tube 160 has a lumen extending therethrough, extending from proximal edge 164 to distal edge 166. Second fixed distal tube 170 is attached within outer tube 152 adjacent the optional end cap 158 and forms a pocket for the second floating channel 156. Distal tube 170 has a lumen 171 extending therethrough, extending from proximal edge 174 to distal edge 176. Preferably, distal edge 166 and 176 are substantially flush with the distal edge of end cap 158. First floating channel 154 has a distal end 154b which in the position of FIG. 13A is fully within the first fixed distal tube 160. Upon bending of the outer tube 152 in one direction, the second floating channel 156 moves distally to the position of FIG. 13B. Upon additional bending, the floating channel 156 can extend beyond the distal edge 176 of the first fixed distal tube 170 (and beyond the distal edge of the outer tube 152 or end cap 158) as shown in FIG. 13C. In bending of the outer tube 152 in the opposite direction of FIG. 13C, the second floating channel 156 remains within the lumen of the second fixed distal tube while the distal end 154b of second floating channel 154 extends distally beyond the distal edge 166 of first fixed distal tube 160 (and beyond the distal edge of the outer tube 152 or end cap 158).

Stated another way, the floating channels 154, 156 are unconstrained within outer tube (catheter) 152 and take the shortest path when the outer tube 152 is bent. Thus, the movement readjusts their position to adjust for the length difference on bending of the outer tube 152. Note the floating channels 154, 156 can also slightly rotate during bending of the outer tube 152 to compensate for stress applied to the floating channels during bending. Consequently, this prevents the eccentric positioned channels from being stretched on the outer portion of the curvature and buckling on the inner portion of the curvature. The floating channels can move around within lumen 153 of outer tube 152 and take any shape to accommodate bending to increase the flexibility of the device.

Note that in FIG. 15A the outer tube 152 is sufficiently bent in a first direction so that first floating channel 154 on the inside curvature of the outer tube 152 is advanced distally beyond distal tube 160. If sufficiently bent in a second opposite direction, the second floating channel 156 on the inside curvature of the outer tube 152 would extend beyond the distal tube 170.

The fixed distal tubes 160, 170 which form pockets for the respective floating channels 154, 156 are dimensioned so their length exceeds the largest extent of movement in response to the greatest curvature of the outer tube 152 as a result of bending of the outer tube 152 during use. This ensures that the floating channels 154, 156 will not retract out of the proximal end of the respective fixed distal tubes 160, 170, In a preferred embodiment, the length of the distal tubes 160, 170 is between about 1.5 cm to about 3 cm, and preferably about 2 cm. Other dimensions are also contemplated.

In the alternate embodiment of FIG. 12, the distal end of the first flexible channel (tube) 180 is positioned within the first fixed distal tube 182 and secured thereto such as by bonding or welding or other attachment methods. It can terminate in any fixed position within the distal tube 182, and in the illustrated embodiment, terminates at the distal end of the distal tube 182

The proximal end of the flexible channel 180 is positioned within a first fixed proximal tube similar to proximal tube 169 discussed above, and secured thereto such as by bonding or welding or other attachment methods. It can terminate in any fixed position within the proximal tube, and in the preferred embodiment terminates at the proximal end of the proximal tube. In this manner, the flexible channel 180 is fixed with respect to the outer tube 152 at its proximal end. It is also fixed with respect to the outer tube 152 at its distal end. It remains unattached in an intermediate portion between the proximal and distal end, e.g., along its length between its two fixed ends, so it can float within the outer tube 152. Similarly, a second flexible channel (not shown) is positioned within a second fixed distal tube and secured thereto such as by bonding or welding or other attachment methods. It can terminate in any fixed position within the distal tube, and like flexible channel 180, can terminate at the distal end of the distal tube. The proximal end of second flexible channel is positioned within a second fixed proximal tube like proximal tube 179 and secured thereto such as by bonding or welding or other attachment methods. It can terminate in any fixed position within the proximal tube, and like flexible channel 180, can terminate at the proximal end of the proximal tube. In this manner, the second flexible channel is fixed with respect to the outer tube 152 at its proximal end and at its distal end. However, it remains unattached in an intermediate portion between the proximal and distal end, e.g., along its length between its two fixed ends, so it can float within the outer tube 152.

The above described first and second flexible guides or tool channels are inserted through the ports of the catheter and extend through the floating channels 154, 156 (or 180), to emerge out the distal ends into the body space, bending due to their pre-curve, in the same manner as in the non-floating embodiments discussed above. That is, flexible guides identical to the flexible guides described above, e.g., guides 12, 14, 114, 116, etc., are inserted through the floating channels 154, 156 in the same manner as described above so that endoscopic working instruments can be inserted therethrough into the body space.

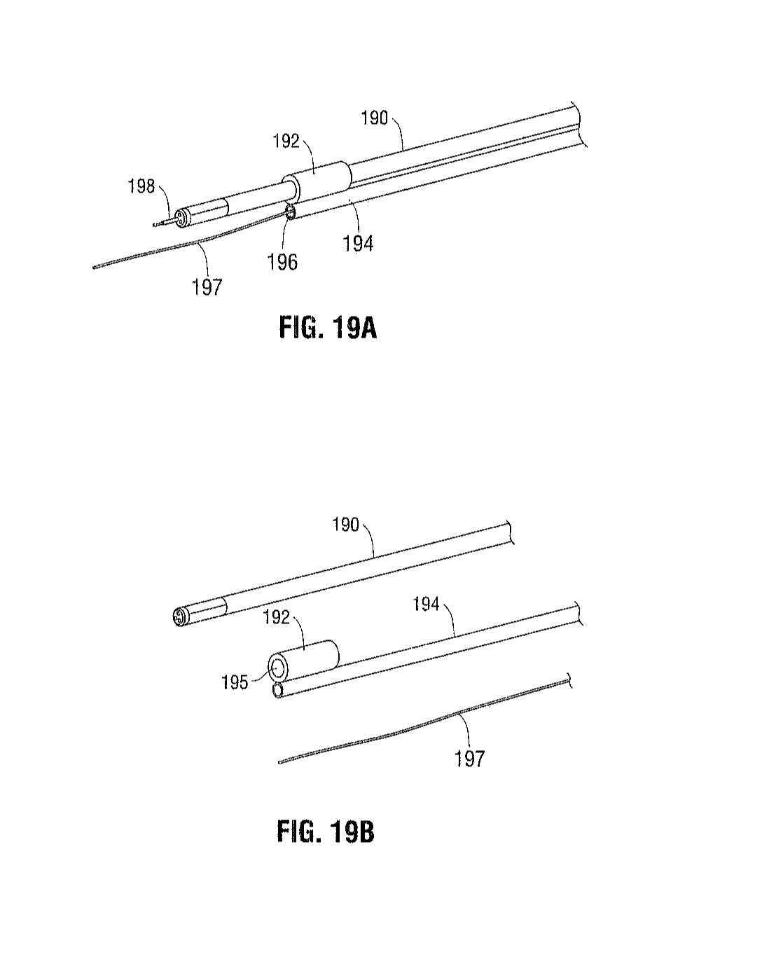

FIGS. 19A and 19B illustrate an alternative embodiment of the system. The system includes an endoscope, a sleeve and a channel. A dissecting instrument 198 is inserted through a working channel of endoscope 190. A short distal overtube 192 has an opening 195 to receive endoscope 190, and is positioned over the endoscope and tightly held thereon such as by a friction fit or other ways of attachment. As shown, the overtube 192 is of a relatively short length overlying a short distal region of the endoscope 190. The overtube 192 has an elongated flexible accessory channel 194 extending therefrom terminating in distal opening 196. The accessory channel 194 can be integral (monolithic) with the overtube 192 so it is a single component or alternatively a separate component attached to the overtube 192. The channel 194 is not attached to the endoscope other than through the attachment of the overtube 192. The accessory channel 194 is of sufficient length so that a proximal end of the channel 194 extends outside the patient for access and/or manipulation by the clinician. An instrument 197 such as one carrying a thread to aid retraction can be inserted through the channel 194. Alternatively, a flexible tube (flexible guide) can be inserted to receive an endoscopic instrument similar to the flexible tubes described above.

The working instruments utilized with any of the systems disclosed herein can include graspers for example. A dissecting/cutting instrument can be inserted through the flexible guide in the floating channel, or alternatively inserted through a working channel of the endoscope. Thus, various working instruments can be inserted through the flexible channels and endoscope channel(s).

Note the endoscopic instruments can be used for partial tissue resection, for example, submucosal or subserosal resection. The endoscopic instruments could also be utilized for full thickness tissue resection. The instruments enable removal of the lesion with healthy tissue margins, thereby providing a complete, en-block removal of the pathological lesion.

In some embodiments the flexible guides and/or endoscopic instruments can be robotically controlled.

Without intending to be limited to any theory or mechanism of action, the above teachings were provided to illustrate a sampling of all possible embodiments rather than a listing of the only possible embodiments. As such, it should be appreciated that there are several variations contemplated within the skill in the art that will also fall into the scope of the claims.

We claim:

1. A system for performing minimally invasive procedures in a body lumen of a patient, the system comprising;
a flexible catheter having a proximal end, a distal end, a first lumen and a second lumen, the first lumen terminating in a first distal opening at a distalmost end of the catheter and the second lumen terminating in a second distal opening at a distalmost end of the catheter, the first and second distal openings being at the terminal end of the flexible catheter;
a first flexible guide being a separate component from the catheter and removably insertable into the flexible catheter and movable axially through the first lumen of the flexible catheter, the first flexible guide having a first channel extending therethrough configured and dimensioned to receive a first endoscopic tool for axial movement therein, the first channel terminating in a first opening, the first flexible guide having a first longitudinal axis and a tube distal portion movable to a pre-shaped curved position with respect to the first longitudinal axis when exposed from the flexible catheter; and
a second flexible guide being a separate component from the catheter and removably insertable into the flexible catheter and movable axially through the second lumen of the flexible catheter, the second flexible guide having a second channel extending therethrough configured and dimensioned to receive a second endoscopic tool for axial movement therein, the second channel terminating in a second opening, the second flexible guide having a second longitudinal axis and a tube distal portion movable to a pre-shaped curved position with respect to the second longitudinal axis when exposed from the flexible catheter.

2. The system of claim 1, wherein the first lumen is in a first flexible tube and the second lumen is in a second flexible tube, the first and second flexible tubes are fixed at a proximal portion to the flexible catheter and configured to float within the flexible catheter such that at least an intermediate portion of the first flexible tube moves radially within the first lumen.

3. The system of claim 1, wherein the first and second flexible guides have different indicators to differentiate the first and second flexible guides.

4. The system of claim 1, wherein the first and second flexible guides are removably insertable through respective first and second proximal ports of the flexible catheter and are independently rotatable and axially movable within the catheter, the first proximal port communicating with the first lumen and the second proximal port communicating with the second lumen.

5. The system of claim 1, wherein one or both of the first flexible guide and second flexible guide includes a valve at a proximal portion to accommodate the respective endoscopic tool without losing insufflation.

6. The system of claim 1, wherein the first and second flexible guides are unattached to the catheter during manipulation by a user from a proximal region, the proximal region protruding proximally from the proximal end of the catheter.

7. The system of claim 1, wherein the first and second flexible guides are composed of shape memory material.

8. The system of claim 2, further comprising an endoscope positionable in the catheter, wherein the endoscope floats within the catheter to increase the flexibility of the catheter.

9. The system of claim 1, wherein the distal portions of the first and second flexible guides are in a straighter condition positioned within confines of the catheter and automatically return to the pre-shaped curved position when exposed from the catheter, and the first and second flexible guides are retractable proximally into the catheter.

10. The system of claim 1, wherein a diameter of a proximal region of the first flexible guide is greater than a diameter of a distal region to provide a stop for distal insertion of the first flexible guide within the first lumen.

11. The system of claim 1, further comprising a first seal within the first lumen and a second seal within the second lumen, the first and second seals limit gas leakage in a space between the flexible guides and the respective lumen.

12. The system of claim 11, wherein the catheter has a third lumen to receive the endoscope, the third lumen having a third seal to limit gas leakage in a space between the endoscope and the third lumen.

13. The system of claim 1, further comprising a blocking member external of the catheter to limit gas leakage.

14. The system of claim 1, further comprising a first tubular support positioned within the flexible catheter, wherein the first flexible guide is unattached to the flexible catheter at a distal end so the first flexible guide telescopes within the first tubular support as the flexible catheter is bent a sufficient amount and a second tubular support is positioned within the flexible catheter, wherein the second flexible guide is unattached to the flexible catheter at a distal end so the second flexible guide telescopes with respect to the second tubular support as the catheter is bent a sufficient amount.

15. A kit for performing minimally invasive procedures in a body lumen of a patient, the kit comprising:
a) first flexible guide being a separate component from and removably insertable into a flexible catheter and movable axially through the flexible catheter, the first flexible guide having a first channel extending therethrough configured and dimensioned to receive a first endoscopic tool for axial movement therein, the first channel terminating in a first opening, the first flexible guide having a first longitudinal axis and a tube distal portion movable to a pre-shaped curved position with respect to the first longitudinal axis when exposed from the flexible catheter; and
b) a second flexible guide being a separate component from the catheter and removably insertable into the flexible catheter and movable axially through the flexible catheter, the second flexible guide having a second channel extending therethrough configured and dimensioned to receive a second endoscopic tool for axial movement therein, the second channel terminating in a second opening, the second flexible guide having a second longitudinal axis and a tube distal portion movable to a pre-shaped curved position with respect to the second longitudinal axis when exposed from the flexible catheter, the second flexible guide being different than the first flexible guide.

16. The kit of claim 15, wherein the first and second flexible guides are different in that the pre-shaped curve of the first flexible guide is a different configuration than the pre-shaped curve of the second flexible guide.

17. The kit of claim 15, wherein the pre-shaped curve of the first flexible guide has a greater radius of curvature than the pre-shaped curve of the second flexible guide.

18. A system for performing minimally invasive procedures in a body lumen of a patient, the system comprising;
an endoscope having a working channel;
an overtube having an opening dimensioned to receive the endoscope; and
an elongated channel extending from the overtube and extending external of an outer wall of the endoscope, the elongated channel dimensioned to receive an instrument therethrough.

19. The system of claim 18, wherein the elongated channel is a separate component extending from the overtube.

20. The system of claim 18, wherein the elongated channel and overtube are monolithic.
